(19) United States
(12) Patent Application Publication
(10) Pub. No.: US 2023/0355618 A1
(43) Pub. Date: Nov. 9, 2023

(54) RAF INHIBITOR FOR TREATING LOW GRADE GLIOMA

(71) Applicants: Day One Biopharmaceuticals, Inc., Brisbane, CA (US); The Brigham and Women's Hospital, Inc., Boston, MA (US); Dana-Farber Cancer Institute Inc., Boston, MA (US)

(72) Inventors: Karen D. WRIGHT, Boston, MA (US); Daphne Adele HAAS-KOGAN, Boston, MA (US); Samuel c. BLACKMAN, Eastsound, WA (US)

(21) Appl. No.: 18/035,478

(22) PCT Filed: Nov. 5, 2021
(86) PCT No.: PCT/US2021/058337

§ 371 Date: May 4, 2023

Related U.S. Application Data
(60) Provisional application No. 63/110,724, filed on Nov. 6, 2020.
(60) Provisional application No. 63/138,285, filed on Jan. 15, 2021.

(51) Int. Cl. A61K 31/506; A61K 45/06; A61P 35/00

(52) U.S. Cl. CPC A61K 31/506; A61K 45/06; A61P 35/00

(57) ABSTRACT

Described herein are methods and compositions for treating gliomas such as pediatric low grade glioma. In some embodiments, a herein described method of treating pediatric low grade glioma comprises administering to a subject in need thereof (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof. In some embodiments, Compound A or a pharmaceutically acceptable salt thereof is administered in an amount that is equivalent to about 400 mg/m² to about 600 mg/m² of Compound A per week. In some embodiments, the subject is less than 20 years of age.

### CROSS-REFERENCE

This application claims the benefit of U.S. Provisional Patent Application No. 63/110,724 filed on Nov. 6, 2020, and U.S. Provisional Patent Application No. 63/138,285, filed on Jan. 15, 2021, the entire contents of each of which are incorporated herein by reference.

### STATEMENT AS TO FEDERALLY SPONSORED RESEARCH

This invention was made with government support under award number P50 CA 165962 as part of the SPORE grant for Targeted Therapies in Gliomas by National Cancer Institute of the National Institutes of Health. The government has certain rights in the invention.

### BACKGROUND

In 2012, there were an estimated 14.1 million cancer cases around the world. This number is expected to increase to 24 million by 2035. Cancer remains the second most common cause of death in the US, accounting for nearly 1 of every 4 deaths. In 2014, there will be an estimated 1,665,540 new cancer cases diagnosed and 585,720 cancer deaths in the US. Although medical advances have improved cancer survival rates, certain patient populations and particular cancers still require further research. In particular, there is an ongoing need for treatment of cancers in pediatric patients.

Each year, approximately 15,500 children under the age of 18 in the United States and 300,000 globally are diagnosed with cancer. Moreover, cancer remains the most common cause of death by disease for children in the United States, accounting for over 1,700 deaths per year. Despite the need for safer and more effective therapies for childhood cancers, new drugs for pediatric patients are rare. There are no approved therapies and no standard of care for pediatric patients with relapsed or progressive low-grade glioma, or pLGG, the most common brain tumor diagnosed in children. Thus, there exists an unmet need for new and more effective treatment for pediatric cancer patients.

### SUMMARY

In one aspect, the present disclosure provides a method of treating a low grade glioma (LGG) in a subject in need thereof comprising, administering (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof to the subject, wherein an initial dose of the Compound A, or a pharmaceutically acceptable salt thereof is equivalent to about 400 mg/m² to about 600 mg/m² of Compound A per week, and wherein the subject is less than 20 years of age. In some embodiments the initial dose of the Compound A, or a pharmaceutically acceptable salt thereof is equivalent to about 500 mg/m² to about 600 mg/m² of Compound A per week. In some embodiments, the initial dose of the Compound A, or a pharmaceutically acceptable salt thereof is equivalent to about 400 mg/m² to about 500 mg/m² of Compound A per week. In some embodiments, the initial dose of the Compound A, or a pharmaceutically acceptable salt thereof is equivalent to about 410 mg/m² to about 430 mg/m² of Compound A per week. In some embodiments, the initial dose of the Compound A, or a pharmaceutically acceptable salt thereof is equivalent to about 420 mg/m² of Compound A per week. In some embodiments, the initial dose of the Compound A, or a pharmaceutically acceptable salt thereof is equivalent to about 530 mg/m² of Compound A per week.

In one aspect, the present disclosure provides a method of treating a low grade glioma (LGG) in a subject in need thereof comprising, administering to the subject (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof in an amount sufficient to achieve in the subject a maximum observed blood plasma concentration (Cmax) of Compound A of at least 2000 ng/mL, and wherein the subject is less than 20 years of age. In some embodiments, the Compound A or a pharmaceutically acceptable salt thereof is administered in an amount sufficient to achieve in the subject a Cmax of Compound A of 2000 ng/mL to 8000 ng/mL.

In one aspect, the present disclosure provides a method of treating a low grade glioma (LGG) in a subject in need thereof comprising, administering to the subject (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof in an amount sufficient to achieve an area under the concentration curve (AUCₛₛ) of Compound A of at least about 400,000 ng*h/ml, wherein the subject is less than 20 years of age. In some embodiments, the Compound A or a pharmaceutically acceptable salt thereof is administered in an amount sufficient to achieve in the subject an (AUCₛₛ) of Compound A of 400,000 ng*h/ml to 1,600,000 ng*h/ml.

In one aspect, provided herein is a method of treating a low grade glioma (LGG) in a subject in need thereof comprising: administering to the subject (i) (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof, in combination with (ii) one or more therapeutic agents for treating a skin-related condition or disorder and wherein the subject is less than 20 years of age. In some embodiments, the one or more therapeutic agents are administered on pigmented skin.

In one aspect, described herein is a method of treating a low grade glioma (LGG) in a subject in need thereof by administering Compound A or a pharmaceutically acceptable salt thereof. In some embodiments, the LGG is a radiographically recurrent or radiographically progressive disease. In some embodiments, the Compound A or a pharmaceutically acceptable salt thereof is (R)-2-(1-(6-amino-5-chloropyrimidine-4-carboxamido)ethyl)-N-(5-chloro-4-(trifluoromethyl)pyridin-2-yl)thiazole-5-carboxamide. In some embodiments, the Compound A or a pharmaceutically acceptable salt thereof is administered as a liquid suspension. In some embodiments, the Compound A or a pharmaceutically acceptable salt thereof is administered as a tablet. In some embodiments, the Compound A or a pharmaceutically acceptable salt thereof is administered as a single dose per week. In some embodiments, the Compound A or a pharmaceutically acceptable salt thereof is administered 2-4 doses a week. In some embodiments, the Compound A or a pharmaceutically acceptable salt thereof is administered for a period of at least 24 months. In some embodiments, the subject is 20 years of age or less. In some embodiments, the subject is 15 years of age or less. In some embodiments, the subject has a body surface area (BSA) of from 0.5 m² to about 2.0 m². In some embodiments, the subject has a BSA of from 0.5 m² to about 1.5 m². In some embodiments, the LGG has one or more of the following mutations: RAS positive mutation, RAF positive mutation, MEK positive mutation, and ERK positive mutation. In some embodiments, the LGG has a BRAF mutation. In some embodiments, the BRAF mutation is a non-V600 BRAF mutation. In some embodiments, the subject is identified having one or more of the following wild-type fusions: KIAA1549:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF21:BRAF, FXR1:BRAF, RNF130:BRAF, BRAF:MACF1, TMEM106B:BRAF, PPC1CC:BRAF, CUX1:BRAF, SRGAP3:RAF1, QK1:RAF1, FYCO:RAF1, ATG7:RAF1, and NFIA:RAF1. In some embodiments, the subject is identified having KIAA1549:BRAF wild-type fusion.

In another aspect, the present disclosure provides a method of treating a low grade glioma (LGG) in a subject in need thereof, as described herein, which further comprises the administration of Compound A or a pharmaceutically acceptable salt thereof at a maximum dose. In some embodiments, the maximum dose of Compound A or a pharmaceutically acceptable salt thereof, is 600 mg. In some embodiments, the maximum dose of Compound A or a pharmaceutically acceptable salt thereof, is 600 mg orally (PO) once a week.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates Phase 1 trial data with pLGG patients that had a complete (100% reduction) or partial response (>50% reduction in the bi-dimensional measurement of the tumor) to treatment with Compound A. Five of the eight patients with a RAF fusion had either a complete response or a partial response per RANO criteria, defined as ≥50% decrease, compared with baseline. Two of eight patients with a RAF fusion had prolonged stable disease. One patient with a RAF fusion did not respond to Compound A. One patient with an NF1-associated pLGG did not respond to Compound A.

FIG. 2. illustrates Phase 1 trial data of individual pLGG patient responses to Compound A over time. Shrinkage in lesion size was observed in six of nine patients in the first radiologic images obtained after initiation of Compound A dosing. The median time to response was 10.5 weeks. Two patients achieved a complete response that was maintained throughout the dosing period of up to two years. Three patients had a partial response, two achieved prolonged stable disease, and two did not achieve a response.

FIG. 3 illustrates Phase 2 trial design of Compound A in pLGG patients. The study includes pediatric patients aged 6 months to 25 years with relapsed or progressive pLGGs harboring an activating BRAF alteration, such as a KIAA1549-BRAF fusion or a BRAF activating mutation, such as V600E. The oral administration of compound A is administered once weekly at a dose of 420 mg/m².

### DETAILED DESCRIPTION OF THE INVENTION

Protein kinases play a critical role in the cell reproduction process. Specifically, the mitogen activated protein kinase (MAPK) signaling pathways consists of a kinase cascade that relays extracellular signals to the nucleus to regulate gene expression and key cellular functions. Gene expression controlled by the Ras/Raf/MEK/ERK signaling pathway regulates fundamental cellular processes including proliferation, differentiation, apoptosis, and angiogenesis. These diverse roles of Ras/Raf/MEK/ERK signaling are aberrantly activated in various types of cancer. Mutations in genes within this pathway may lead to constitutively active proteins resulting in increased cell proliferation, and resistance to apoptosis.

Raf (a serine/threonine-protein kinase) is encoded by a gene family consisting of three genes affording three Raf isoform members (B-Raf, C-Raf (Raf-1) and A-Raf). Each of these proteins share highly conserved amino-terminal regulatory regions and catalytic domains at the carboxy terminus. Although each isoform plays a role in the Ras/Raf/MEK/ERK pathway, B-Raf has been shown to be the main activator of MEK. B-Raf is recruited by Ras:GTP to the intracellular cell membrane where B-Raf becomes activated. In turn, B-Raf is responsible for activation of MEK1/2 and MEK1/2 activate ERK1/ERK2. Mutations in the B-Raf gene allow for B-Raf to signal independently of upstream signals. As a result, mutated B-Raf protein (such as V600E) causes excessive downstream signaling of MEK and ERK. This leads to excessive cell proliferation and survival and oncogenesis. Overactivation of the signaling cascade by mutated B-Raf has been implicated in multiple malignancies. B-Raf specific inhibitors (such as vemurafenib) are in fact, showing promise for the treatment of melanomas that express mutant B-Raf V600E.

Gliomas are histologically defined based on whether they exhibit primarily astrocytic or oligodendroglial morphology, and are graded by cellularity, nuclear atypia, necrosis, mitotic figures, and microvascular proliferation all features associated with biologically aggressive behavior. Astrocytomas are of two main types high-grade and low-grade. High-grade tumors grow rapidly, are well-vascularized, and can easily spread through the brain. Low-grade astrocytomas are usually localized and grow slowly over a long period of time. High-grade tumors are much more aggressive, require very intensive therapy, and are associated with shorter survival lengths of time than low grade tumors. The majority of astrocytic tumors in children are low-grade, whereas the majority in adults are high-grade. These tumors can occur anywhere in the brain and spinal cord. Some of the more common low-grade astrocytomas are: Juvenile Pilocytic Astrocytoma (JPA), Fibrillary Astrocytoma Pleomorphic Xantroastrocytoma (PXA) and Desembryoplastic Neuroepithelial Tumor (DNET). The two most common high-grade astrocytomas arc Anaplastic Astrocytoma (AA) and Glioblastoma Multiforme (GBM).

Pediatric low-grade gliomas (PLGGs) encompass a heterogeneous group of World Health Organization (WHO) grade I and II tumors that collectively represent the most common pediatric brain tumor. They encompass tumors of a variety of histology, such as pilocytic astrocytoma, diffuse astrocytoma, oligodendroglioma and angiocentric glioma. Angiocentric glioma is a WHO grade I tumor that has an indolent clinical course. It arises in the cerebral cortex and shares histological features with astrocytomas and ependymomas. Angiocentric glioma causes medically refractory epileptic seizure in children.

The Ras pathway is implicated in a large number of tumors in adult patients. Low-grade gliomas (LGG) are the most common brain tumor in children, and the majority have abnormal signaling through the RAS/RAF pathway. Complete resection is often not feasible in many patients, and incompletely resected LGG have a high rate of progression and recurrence. Best currently available therapies have limited efficacy, and the long-term burden of disease and treatment-related morbidity is significant. Results from the largest randomized phase III trial to date for children with LGG showed a 5-year event free survival of only 47%.

The present disclosure provides a method of treating gliomas such as pediatric low-grade gliomas by administering Compound A, or a pharmaceutically acceptable salt or solvate thereof. It was discovered that a dose regimen based on the patient's body surface area (e.g., mg/m²) is suitable for the described method. In some embodiments, a dosing regimen based on the patient's body surface area (e.g., mg/m²) is more suitable than a dosing regimen based on the patient's body weight (e.g., mg/kg). It was also discovered that it can be beneficial to initiate the treatment at a certain dose amount, e.g., higher than 400 mg/m² of Compound A per week.

Terms used herein shall be accorded the following defined meanings, unless otherwise indicated.

### Definitions

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

As used herein, the term “Raf kinase” refers to any one of a family of serine/threonine-protein kinases. The family consists of three isoform members (B-Raf, C-Raf (Raf-1), and A-Raf). Raf protein kinases are involved in the MAPK signaling pathway consisting of a kinase cascade that relays extracellular signals to the nucleus to regulate gene expression and key cellular functions. Unless otherwise indicated by context, the term “Raf kinase” is meant to refer to any Raf kinase protein from any species, including, without limitation. In one aspect, the Raf kinase is a human Raf kinase.

The term “Raf inhibitor” or “inhibitor of Raf’ is used to signify a compound which is capable of interacting with one or more isoform members (B-Raf, C-Raf (Raf-1) and/or A-Raf) of the serine/threonine-protein kinase, Raf including mutant forms. Some examples of Raf mutant forms include, but are not limited to B-Raf V600E, B-Raf V600D, B-Raf V600K, B-Raf V600E+T5291 and/or B-Raf V600E+G468A.

In some embodiments, the Raf kinase is at least about 50% inhibited, at least about 75% inhibited, at least about 90% inhibited, at least about 95% inhibited, at least about 98% inhibited, or at least about 99% inhibited. In some embodiments, the concentration of Raf kinase inhibitor required to reduce Raf kinase activity by 50% is less than about 1 μM, less than about 500 nM, less than about 100 nM, less than about 50 nM, less than about 25 nM, less than about 10 nM, less than about 5 nM, or less than about 1 nM.

In some embodiments, such inhibition is selective for one or more Raf isoforms, i.e., the Raf inhibitor is selective for one or more of B-Raf (wild type), mutant B-Raf, A-Raf, and C-Raf kinase. In some embodiments, the Raf inhibitor is selective for B-Raf (wild type), B-Raf V600E, A-Raf and C-Raf. In some embodiments, the Raf inhibitor is selective for B-Raf (wild type), B-Raf V600E, A-Raf and C-Raf. In some embodiments, the Raf inhibitor is selective for B-Raf (wild type), B-Raf V600D, A-Raf and C-Raf. In some embodiments, the Raf inhibitor is selective for B-Raf (wild type), B-Raf V600K, and C-Raf. In some embodiments, the Raf inhibitor is selective for more than B-Raf V600. In some embodiments, the Raf inhibitor is selective for more than B-Raf V600E.

In some embodiments, the Raf inhibitor is selective for B-Raf and C-Raf kinases. In some embodiments, the Raf inhibitor is selective for B-Raf (wild type), B-Raf V600E and C-Raf. In some embodiments, the Raf inhibitor is selective for B-Raf (wild type), B-Raf V600D and C-Raf. In some embodiments, the Raf inhibitor is selective for B-Raf (wild type), B-Raf V600K and C-Raf. In some embodiments, the Raf inhibitor is selective for mutant B-Raf. In some embodiments, the Raf inhibitor is selective for mutant B-Raf V600E. In some embodiments, the Raf inhibitor is selective for mutant B-Raf V600D. In some embodiments, the Raf inhibitor is selective for mutant B-Raf V600K.

The term “pan-Raf inhibitor” is a Raf inhibitor that inhibits more than the B-Raf V600 isoform of Raf proteins.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. In some embodiments, “about” refers to a range of up to 10% of a given value. In some embodiments, “about” refers to a range of up to 5% of a given value. Alternatively, particularly with respect to biological systems or processes, the term can mean within an order of magnitude, within 5-fold, or within 2-fold, of a value.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below. As used herein, the terms “treatment,” “treat,” and “treating” are meant to include the full spectrum of intervention for the cancer from which the subject is suffering, such as administration of the combination to alleviate, slow, stop, or reverse one or more symptoms of the cancer and to delay the progression of the cancer even if the cancer is not actually eliminated. Treatment can include, for example, a decrease in the severity of a symptom, the number of symptoms, or frequency of relapse, e.g., the inhibition of tumor growth, the arrest of tumor growth, or the regression of already existing tumors.

The term “therapeutically effective amount” as used herein to refer to an amount effective at the dosage and duration necessary to achieve the desired therapeutic result. A therapeutically effective amount of the composition may vary depending on factors such as the individual's condition, age, sex, and weight, and the ability of the protein to elicit the desired response of the individual. A therapeutically effective amount is also an amount that exceeds any toxic or deleterious effect of the composition that would have a beneficial effect on the treatment.

The term “subject”, as used herein, means a mammal, and “mammal” includes, but is not limited to a human. In some embodiments, the subject has received treatment prior to initiation of treatment according to the method of the disclosure. In some embodiments, the subject is at risk of developing or experiencing a recurrence of a cancer.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a 13C- or 14C-enriched carbon are within the scope of the disclosure.

Certain compounds described herein may exist in tautomeric forms, and all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

### RAF Inhibitors

Compounds capable of inhibiting the activity of a Raf kinase maybe be used in the methods of the instant disclosure. In some embodiments, the Raf inhibitor inhibits more isoforms of Raf kinase proteins than B-Raf V600. In some embodiment, the Raf inhibitor inhibits more isoforms of Raf kinase proteins than B-Raf V600E. In some embodiments, the Raf inhibitor inhibits B-Raf (wild-type), mutant B-Raf, A-Raf, and C-Raf. In some embodiments, the Raf inhibitor is selective for B-Raf (wild-type), B-Raf V600E, A-Raf and/or C-Raf. In some embodiments, the Raf inhibitor is selective for B-Raf (wild-type), B-Raf V600K, A-Raf and/or C-Raf. In some embodiments, the Raf inhibitor is selective for B-Raf (wild-type), B-Raf V600D, A-Raf and/or C-Raf. In some embodiments, the Raf inhibitor is selective for B-Raf (wild-type), B-Raf V600K, and C-Raf. In some embodiments, the Raf inhibitor is selective for B-Raf (wild-type), B-Raf V600E and C-Raf. In some embodiments, the Raf inhibitor is selective for B-Raf (wild-type), B-Raf V600D and C-Raf. In some embodiments, the Raf inhibitor is selective for B-Raf (wild-type), B-Raf V600K and C-Raf. In some embodiments, the Raf inhibitor is selective for mutant B-Raf. In some embodiments, the Raf inhibitor is selective for mutant B-Raf V600E. In some embodiments, the Raf inhibitor is selective for mutant B-Raf V600D. In some embodiments, the Raf inhibitor is selective for mutant B-Raf V600K.

Raf inhibitors can be assayed in vitro or in vivo for their ability to bind to and/or inhibit Raf kinases. In vitro assays include biochemical FRET assays to measure the phophorylation of MEK by Raf kinases as a method for quantifying the ability of compounds to inhibit the enzymatic activity of Raf kinases. The compounds also can be assayed for their ability to affect cellular or physiological functions mediated by Raf kinase activity. For example in vitro assays quantitate the amount of phosphor-ERK in cancer cells. Assays for each of these activities are known in the art.

In some embodiments, the pharmaceutical compositions also comprise one or more fillers (e.g., mannitol, celluloses, calcium carbonate, starches, sugars (e.g., dextrose, lactose or the like)) in concentrations of at least about 10 wt % by weight of the composition; a sweetener (e.g. sucralose, sorbitol, saccharin, fructose, aspartame, or a combination thereof) in a concentration of about 10% or less by weight of this composition; a disintegrant (e.g., croscarmellose sodium, sodium starch glycolate, or a combination thereof) in concentrations of about 10 wt % or less by weight of the composition; optionally a wetting agent (e.g., sodium lauryl sulfate, SLS) in concentrations of about 10 wt % or less by weight of the composition; a glidant (e.g., colloidal silicon dioxide, talc, or a combination thereof) in concentrations of about 2 wt % or less by weight of the composition; and a lubricant (e.g., magnesium stearate, stearic acid, hydrogenated oil, sodium stearyl fumarate, or any combination thereof) in concentrations of about 5 wt % or less by weight of the composition.

Such pharmaceutical compositions can optionally comprise one or more colorants, fragrances, and/or flavors to enhance its visual appeal, taste, and scent.

In other embodiments, the present invention provides a pharmaceutical composition in the form of a powder composition, as described above, which can also be formulated into solid unit dose forms for the treatment of the various diseases. The present invention therefore also contemplates novel dosage forms such as granules, pellets, mini-tablets and other solid dose forms which overcome the problems described above with respect to dosing inaccuracies, in particular, for pediatric patients. These stable, solid unit dose forms can have any shape, including oval, spherical, cylindrical, elliptical, cubical, square, or rectangular among others. Tablets or mini-tablets may have flat, shallow, standard, deep convex, or double deep convex faces or combinations thereof. In some embodiments, Compound A or pharmaceutically acceptable salt or solvate thereof is formulated to have a strength of 10 to 500 mg per tablet. In some embodiments, Compound A or pharmaceutically acceptable salt or solvate thereof is formulated to have a strength of 10 to 50 mg, 25 to 75 mg, 50 to 100 mg, 75 to 125 mg, 125 to 175 mg, or 150 to 250 mg per tablet. In some embodiments, Compound A or pharmaceutically acceptable salt or solvate thereof is formulated to have a strength of 20 mg per tablet. In some embodiments, Compound A or pharmaceutically acceptable salt or solvate thereof is formulated to have a strength of 100 mg per tablet. In some embodiments, Compound A or pharmaceutically acceptable salt or solvate thereof is formulated to have a strength of 10, 20, 25, 50, 75, 100, 150 or 200 mg per tablet. In some embodiments, the dose strength of the tablet is based on the free base of Compound A.

In other embodiments, the present invention provides a pharmaceutical composition that can be formulated into a tablet. In some embodiments, the tablet comprises compound A or a pharmaceutically acceptable salt or solvate thereof. In some embodiments, the tablet may further comprise an acceptable excipient. In some embodiments, the acceptable excipient may include, but are not limited to, one or more of microcrystalline cellulose colloidal silicon dioxide, magnesium stearate, vinylpyrrolidone-vinyl acetate copolymer (copovidone), sodium croscarmellose and Opradry®. In some embodiments, the tablets are coated with different colors.

In one aspect, the pharmaceutical composition can be formulated into a unit dose form, for example, a capsule, a sachet, and the like, containing at least one or more mini-tablets to simplify the administration of the pharmaceutical composition. In some embodiments, the unit dose can include a capsule or a packet containing at least one mini-tablet, or a plurality of mini-tablets as provided above and in the descriptions below. In another embodiment, the unit dose can include a pouch, a packet or sachet containing a specific dose of substantially amorphous or amorphous Compound A, or a pharmaceutically acceptable salt or solvate thereof, in powder form.

In some embodiments, described herein is a Raf inhibitor that is (R)-2-(1-(6-amino-5-chloropyrimidine-4-carboxamide)ethyl)-N-(5-chloro-4-(trifluoromethyl)pyridin-2-yl)thiazole-5-carboxamide (Compound A), or a pharmaceutically acceptable salt or solvate thereof. The structure of Compound A is illustrated below:

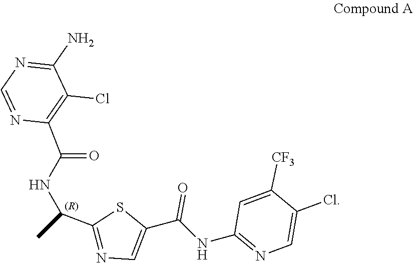

Compound A, or a pharmaceutically acceptable salt or solvate thereof, is described in U.S. Pat. No. 8,293,752B2. Compound A is also called DAY101, formally TAK-580, B1113024, or MLN2480. Compound A is also called tovorafenib. In some embodiments, the Raf inhibitor is (R)-2-(1-(6-amino-5-chloropyrimidine-4-carboxamide)ethyl)-N-(5-chloro-4-(trifluoromethyl)pyridin-2-yl)thiazole-5-carboxamid. In some embodiments, the Raf inhibitor is Compound A, or a pharmaceutically acceptable salt or solvate thereof. In some embodiments, the Raf inhibitor is a pharmaceutically acceptable salt of Compound A. In some embodiments, the Raf inhibitor is a solvate of Compound A. In some embodiments, the Raf inhibitor is a crystalline form of Compound A. In some embodiments, the Raf inhibitor is a hydrate of Compound A. In some embodiments, the Raf inhibitor is a crystalline form of Compound A. In some embodiments, the Raf inhibitor is

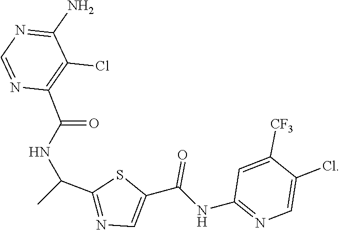 [SMILES: CC(NC(=O)c1ncnc(N)c1Cl)c1ncc(C(=O)Nc2cc(C(F)(F)F)c(Cl)cn2)s1]

In some embodiments, described herein is a pharmaceutically acceptable salt of Compound A. Suitable pharmaceutically acceptable salts include those described in, for example, S. M. Berge et al., d J. Pharmaceutical Sciences, 1977, 66, 1-19, incorporated herein by reference. Pharmaceutically acceptable salts of compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alky)₄ salts. The present disclosure also envisions the quaternization of any basic nitrogen-containing groups. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Dosing

Suitable daily dosages of inhibitors of Raf kinase can generally range, in single or divided or multiple doses, from about 10% to about 100% of the maximum tolerated dose as a single agent. In some embodiments, the suitable dosages are from about 15% to about 100% of the maximum tolerated dose as a single agent. In some embodiments, the suitable dosages are from about 25% to about 90% of the maximum tolerated dose as a single agent. In some other embodiments, the suitable dosages are from about 30% to about 80% of the maximum tolerated dose as a single agent. In some other embodiments, the suitable dosages are from about 40% to about 75% of the maximum tolerated dose as a single agent. In some other embodiments, the suitable dosages are from about 45% to about 60% of the maximum tolerated dose as a single agent. In some embodiments, suitable dosages are about 10%, about 15%, about 20%, about 25%, about 30%, about 35%, about 40%, about 45%, about 50%, about 55%, about 60%, about 65% o, about 70%, about 75%, about 80%, about 85%, about 90%, about 95%, about 100%, about 105%, or about 110% of the maximum tolerated dose as a single agent.

A suitable dosage of a Raf inhibitor may be taken at any time of the day or night. In some embodiments, a suitable dosage of a selective inhibitor of Raf inhibitor is taken in the morning. In some other embodiments, a suitable dosage of a Raf inhibitor is taken in the evening. In some other embodiments, a suitable dosage of a Raf inhibitor is taken both in the morning and the evening. It will be understood that a suitable dosage of a Raf inhibitor may be taken with or without food. In some embodiments a suitable dosage of a Raf inhibitor is taken with a meal. In some embodiments a suitable dosage of a Raf inhibitor is taken while fasting.

Described herein is a method of treating cancer such as glioma (e.g., low grade glioma) that comprises administering a Raf inhibitor. In some embodiments, the method comprises administering Compound A, or a pharmaceutically acceptable salt or solvate thereof. In some embodiments, the method comprises administering a crystalline form of Compound A. In some embodiments, the method comprises administering a salt of Compound A. In some embodiments, the method comprises administering Compound A.

Described herein is a method of treating cancer such as glioma (e.g., low grade glioma) that comprises administering Compound A, or a pharmaceutically acceptable salt or solvate thereof based on a subject's body surface area (BSA), for example mg/m². BSA can be determined by any suitable calculation method. In some embodiments, the BSA is determined by Mosteller Formula (√((height×weight)/3600)). In some embodiments, the BSA is determined at the start of each cycle of administration.

In some embodiments, Compound A, or a pharmaceutically acceptable salt or solvate thereof, is administered in an amount of up to about 600 mg of per dose. In some embodiments, Compound A, or a pharmaceutically acceptable salt or solvate thereof, is administered in an amount of up to about 500 mg, about 600 mg, about 700 mg, about 800 mg, about 900 mg, or about 1000 mg of per dose. In some embodiments, Compound A, or a pharmaceutically acceptable salt or solvate thereof, is administered in an amount of up to about 600 mg of Compound A per dose. In some embodiments, Compound A, or a pharmaceutically acceptable salt or solvate thereof, is administered in an amount of up to about 800 mg of Compound A per dose.

In some embodiments, Compound A, or a pharmaceutically acceptable salt or solvate thereof, is administered in an amount of up to 1200 mg/m² per dose. In some embodiments, Compound A, or a pharmaceutically acceptable salt or solvate thereof, is administered in an amount of up to 1000 mg/m² per dose. In some embodiments, Compound A, or a pharmaceutically acceptable salt or solvate thereof, is administered in an amount of up to 800 mg/m² per dose. In some embodiments, Compound A, or a pharmaceutically acceptable salt or solvate thereof, is administered in an amount of up to 600 mg/m² per dose. In some embodiments, Compound A, or a pharmaceutically acceptable salt or solvate thereof, is administered in an amount of up to 500 mg/m² per dose. In some embodiments, Compound A, or a pharmaceutically acceptable salt or solvate thereof, is administered in an amount of up to 300 mg/m² per dose. In some embodiments, Compound A, or a pharmaceutically acceptable salt or solvate thereof, is administered in an amount of up to 200 mg/m² per dose. Suitable dosages of a Raf inhibitor e.g., Compound A, or a pharmaceutically acceptable salt or solvate thereof, can generally range, in single or divided or multiple doses, from 10 mg/m² to about 1000 mg/m² per dose. In some embodiments, Compound A, or a pharmaceutically acceptable salt or solvate thereof, is administered as a single dose. In some embodiments, Compound A, or a pharmaceutically acceptable salt or solvate thereof, is administered as a divided dose. In some embodiments, Compound A, or a pharmaceutically acceptable salt or solvate thereof, is administered in multiple doses. Other suitable dosages of Compound A, or a pharmaceutically acceptable salt or solvate thereof, can generally range, in single or divided or multiple doses, from about 200 mg/m² to about 800 mg/m² per dose. Other suitable dosages of Compound A, or a pharmaceutically acceptable salt or solvate thereof, can generally range, in single or divided or multiple doses, from about 75 mg/m² to about 200 mg/m² per dose. In some embodiments, the suitable dosages are from about 100 mg/m² to about 200 mg/m² per dose. In some other embodiments, the suitable dosages are from about 150 mg/m² to about 600 mg/m² twice daily. In some embodiments, suitable dosages are about 20 m mg/m², about 25 mg/m², about 30 mg/m², about 35 mg/m², about 40 mg/m², about 45 mg/m², about 50 mg/m², about 55 mg/m², about 60 mg, about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 220 mg/m², about 240 mg/m², about 260 mg/m², about 280 mg/m², about 300 mg/m², about 320 mg/m², about 340 mg/m², about 360 mg/m², about 380 mg/m², about 400 mg/m², about 420 mg/m², about 440 mg/m², about 460 mg/m², about 480 mg/m², about 500 mg/m², about 520 mg/m², about 530 mg/m², about 560 mg/m², about 580 mg/m², about 600 mg/m², about 620 mg/m², about 640 mg/m², about 660 mg/m², about 680 mg/m², about 700 mg/m², about 720 mg/m², about 740 mg/m², about 760 mg/m², about 780 mg/m², about 800 mg/m², about 825 mg/m², about 850 mg/m², about 900 mg/m², about 950 mg/m² about 1000 mg/m², about 1050 mg/m², or about 1100 mg/m² per dose. In some embodiments, the suitable dosage of Compound A, or a pharmaceutically acceptable salt or solvate thereof, is from about 100 mg to about 1000 mg of Compound A per dose. In some embodiments, suitable dosages are about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 530 mg, about 560 mg, about 580 mg, about 600 mg, about 620 mg, about 640 mg, about 660 mg, about 680 mg, about 700 mg, about 720 mg, about 740 mg, about 760 mg, about 780 mg, or about 800 mg per dose In some embodiments, the Compound A, or a pharmaceutically acceptable salt or solvate thereof is administered weekly. In some embodiments, the Compound A, or a pharmaceutically acceptable salt or solvate thereof, is administered once a week. In some embodiments, the dosing is based on the free base form of Compound A.

Compound A, or a pharmaceutically acceptable salt or solvate thereof can be administered to a subject at a starting dose. In some embodiments, the starting dose is about 100 to about 1000 mg/m² of Compound A per week. In some embodiments, the starting dose is about 20 mg/m², about 25 mg/m², about 30 mg/m², about 35 mg/m², about 40 mg/m², about 45 mg/m², about 50 mg/m², about 55 mg/m², about 60 mg, about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 220 mg/m², about 240 mg/m² about 260 mg/m², about 280 mg/m², about 300 mg/m², about 320 mg/m², about 340 mg/m², about 360 mg/m², about 380 mg/m², about 400 mg/m², about 420 mg/m², about 440 mg/m², about 460 mg/m², about 480 mg/m², about 500 mg/m², about 520 mg/m², about 530 mg/m² about 560 mg/m², about 580 mg/m², about 600 mg/m², about 620 mg/m², about 640 mg/m², about 660 mg/m², about 680 mg/m², about 700 mg/m², about 720 mg/m², about 740 mg/m², about 760 mg/m², about 780 mg/m², about 800 mg/m², about 825 mg/m², about 850 mg/m², about 875 mg/m², about 900 mg/m², about 925 mg/m², about 950 mg/m², about 975 mg/m², or about 1000 mg/m² per week. In some embodiments, the Compound A, or a pharmaceutically acceptable salt or solvate thereof is administered once a week. In some embodiments, the Compound A, or a pharmaceutically acceptable salt or solvate thereof is administered 2, 3, 4, 5, 6, or 7 times a week. In some embodiments, the starting dose is about 825 mg/m² per week. In some embodiments, the starting dose is about 660 mg/m² A per week. In some embodiments, the starting dose is about 530 mg/m² per week. In some embodiments, the starting dose is about 420 mg/m² per week. In some embodiments, the starting dose is about 410 mg/m² to about 430 mg/m² per week. In some embodiments, the starting dose is about 400 mg/m² to about 450 mg/m² per week. In some embodiments, the starting dose is about 350 mg/m² to about 450 mg/m² per week. In some embodiments, the starting dose is about 350 mg/m² per week. In some embodiments, the starting dose is about 280 mg/m² per week. In some embodiments, the initial dose is about 600 mg/m² to about 700 mg/m² per week. In some embodiments, the initial dose is about 500 mg/m² to about 550 mg/m² per week. In some embodiments, the initial dose is about 400 mg/m² to about 450 mg/m² per week. In some embodiments, the initial dose is about 400 mg/m² to about 500 mg/m² per week. In some embodiments, the initial dose is about 200 mg/m² to about 300 mg/m² per week. In some embodiments, the initial dose is about 250 mg/m² to about 300 mg/m² per week. In some embodiments, the dosing is based on the free base form of Compound A. In some embodiments, the dosing is administered once a week.

Compound A, or a pharmaceutically acceptable salt or solvate thereof can be administered to a subject at a maintenance dose. In some embodiments, the maintenance dose is equivalent to about 100 to about 1000 mg/m² of Compound A per week. In some embodiments, the maintenance dose is from about 100 to about 800 mg/m² per week. In some embodiments, the maintenance dose is about 20 mg/m², about 25 mg/m², about 30 mg/m², about 35 mg/m², about 40 mg/m², about 45 mg/m², about 50 mg/m², about 55 mg/m², about 60 mg, about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 220 mg/m², about 240 mg/m², about 260 mg/m², about 280 mg/m², about 300 mg/m², about 320 mg/m², about 340 mg/m², about 360 mg/m², about 380 mg/m², about 400 mg/m², about 420 mg/m², about 440 mg/m², about 460 mg/m², about 480 mg/m², about 500 mg/m², about 520 mg/m², about 530 mg/m², about 560 mg/m², about 580 mg/m², about 600 mg/m², about 620 mg/m², about 640 mg/m², about 660 mg/m², about 680 mg/m², about 700 mg/m², about 720 mg/m², about 740 mg/m², about 760 mg/m², about 780 mg/m², about 800 mg/m², about 825 mg/m², about 850 mg/m², about 875 mg/m², about 900 mg/m², about 925 mg/m², about 950 mg/m², about 975 mg/m², or about 1000 mg/m² per week. In some embodiments, the Compound A, or a pharmaceutically acceptable salt or solvate thereof is administered once a week. In some embodiments, the Compound A, or a pharmaceutically acceptable salt or solvate thereof is administered 2, 3, 4, 5, 6, or 7 times a week. In some embodiments, the maintenance dose is about 825 mg/m² per week. In some embodiments, the maintenance dose is about 660 mg/m² per week. In some embodiments, the maintenance dose is about 530 mg/m² per week. In some embodiments, the maintenance dose is about 420 mg/m² per week. In some embodiments, the maintenance dose is about 350 mg/m² per week. In some embodiments, the maintenance dose is about 280 mg/m² per week. In some embodiments, the maintenance dose is about 600 mg/m² to about 700 mg/m² per week. In some embodiments, the maintenance dose is about 500 mg/m² to about 550 mg/m² per week. In some embodiments, the maintenance dose is about 400 mg/m² to about 450 mg/m² per week. In some embodiments, the maintenance dose is about 420 mg/m² per week. In some embodiments, the maintenance dose is about 410 mg/m² to about 430 mg/m² per week. In some embodiments, the maintenance dose is about 350 mg/m² to about 450 mg/m² per week. In some embodiments, the maintenance dose is about 400 mg/m² to about 500 mg/m² per week. In some embodiments, the maintenance dose is about 200 mg/m² to about 300 mg/m² per week. In some embodiments, the maintenance dose is about 250 mg/m² to about 300 mg/m² per week. In some embodiments, the maintenance dose is the same as the initial dose. In some embodiments, the maintenance dose is higher than the initial dose. In some embodiments, the maintenance dose is lower than the initial dose. In some embodiments, the dosing is based on the free base form of Compound A. In some embodiments, the dosing is administered once a week.

Compound A, or a pharmaceutically acceptable salt or solvate thereof can be administered to a subject at a maximum tolerated dose. In some embodiments, the maximum tolerated dose is equivalent to about 100 to about 1200 mg/m² of Compound A per week. In some embodiments, the maximum tolerated dose is about 100 to about 800 mg/m² per week. In some embodiments, the maximum tolerated dose is about 20 mg/m², about 25 mg/m², about 30 mg/m², about 35 mg/m², about 40 mg/m², about 45 mg/m², about 50 mg/m², about 55 mg/m², about 60 mg, about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 220 mg/m², about 240 mg/m², about 260 mg/m², about 280 mg/m², about 300 mg/m², about 320 mg/m², about 340 mg/m², about 360 mg/m², about 380 mg/m², about 400 mg/m², about 420 mg/m², about 440 mg/m², about 460 mg/m², about 480 mg/m², about 500 mg/m², about 520 mg/m², about 530 mg/m², about 560 mg/m², about 580 mg/m², about 600 mg/m², about 620 mg/m², about 640 mg/m², about 660 mg/m², about 680 mg/m², about 700 mg/m², about 720 mg/m², about 740 mg/m², about 760 mg/m², about 780 mg/m², about 800 mg/m², about 825 mg/m², about 850 mg/m², about 875 mg/m², about 900 mg/m², about 925 mg/m², about 950 mg/m², about 975 mg/m², or about 1000 mg/m² per week. In some embodiments, the maximum tolerated dose is about 500 mg/m², about 520 mg/m², about 530 mg/m², about 560 mg/m², about 580 mg/m², or about 600 mg/m² per dose. In some embodiments, the Compound A, or a pharmaceutically acceptable salt or solvate thereof is administered once a week. In some embodiments, the Compound A, or a pharmaceutically acceptable salt or solvate thereof is administered 2, 3, 4, 5, 6, or 7 times a week. In some embodiments, the maximum tolerated dose is about 825 mg/m² per week. In some embodiments, the maximum tolerated dose is about 660 mg/m² per week. In some embodiments, the maximum tolerated dose is about 530 mg/m² per week. In some embodiments, the maximum tolerated dose is about 420 mg/m² per week. In some embodiments, the maximum tolerated dose is about 280 mg/m² per week. In some embodiments, the maximum tolerated dose is about 800 mg/m² to about 1000 mg/m² per week. In some embodiments, the maximum tolerated dose is about 600 mg/m² to about 800 mg/m² per week. In some embodiments, the maximum tolerated dose is about 500 mg/m² to about 550 mg/m² per week. In some embodiments, the maximum tolerated dose is about 400 mg/m² to about 450 mg/m² per week. In some embodiments, the maximum tolerated dose is about 400 mg/m² to about 500 mg/m² per week. In some embodiments, the maximum tolerated dose is about 200 mg/m² to about 300 mg/m² per week. In some embodiments, the maximum tolerated dose is about 250 mg/m² to about 300 mg/m² per week. In some embodiments, the dosing is based on the free base form of Compound A.

In some embodiments, Compound A, or a pharmaceutically acceptable salt or solvate thereof can be chronically administered to a subject. In some embodiments, the chronic administration of Compound A, or a pharmaceutically acceptable salt or solvate thereof is administered once a week. In some embodiments, the chronic dosing administration of Compound A, or a pharmaceutically acceptable salt or solvate thereof, is once a day, once every other day, every third day, or once a week. In some embodiments, the chronic dosing administration of Compound A, or a pharmaceutically acceptable salt or solvate thereof, is at least once a day, at least once every other day, at least every third day, or at least once a week. the chronic dosing administration of Compound A, or a pharmaceutically acceptable salt or solvate thereof, is at most once a day, at most once every other day, at most every third day, or at most once a week. In some embodiments, Compound A, or a pharmaceutically acceptable salt or solvate thereof is chronically administered using an amount as disclosed herein.

In some embodiments, about 280 mg/m² of Compound A, or a pharmaceutically acceptable salt or solvate thereof is chronically administered once a week. In some embodiments, about 350 mg/m² of Compound A, or a pharmaceutically acceptable salt or solvate thereof is chronically administered once a week. In some embodiments, about 420 mg/m² of Compound A, or a pharmaceutically acceptable salt or solvate thereof is chronically administered once a week. In some embodiments, about 530 mg/m² of Compound A, or a pharmaceutically acceptable salt or solvate thereof is chronically administered once a week.

In some embodiments, Compound A, or a pharmaceutically acceptable salt or solvate thereof can be chronically administered to a subject, once a month, twice a month, three times a month, four times a month, five times a month, six times a month, or more. In some embodiments, Compound A, or a pharmaceutically acceptable salt or solvate thereof can be chronically administered to a subject, at least one time a month, two times a month, three times a month, four times a month, five times a month, six times a month. In some embodiments, Compound A, or a pharmaceutically acceptable salt or solvate thereof can be chronically administered to a subject, at most one time a month, at most two times a month, at most three times a month, at most four times a month, at most five times a month, at most six times a month. In some embodiments, Compound A, or a pharmaceutically acceptable salt or solvate thereof is chronically administered using an amount as disclosed herein.

In some embodiments, Compound A, or a pharmaceutically acceptable salt or solvate thereof can be chronically administered to a subject over the course of 30 days, 60 days, 120 days, 180 days, 240 days, 300 days, 360 days, 720 days, 1440 days, 1880 days, or 3600 days. In some embodiments, Compound A, or a pharmaceutically acceptable salt or solvate thereof can be chronically administered to a subject over the course of at least 30 days, at least 60 days, at least 120 days, at least 180 days, at least 240 days, at least 300 days, at least 360 days, at least 720 days, at least 1440 days, at least 1880 days, or at least 3600 days. In some embodiments, Compound A, or a pharmaceutically acceptable salt or solvate thereof can be chronically administered to a subject over the course of at most 30 days, at most 60 days, most 120 days, at most 180 days, at most 240 days, at most 300 days, at most 360 days, at most 720 days, at most 1440 days, at most 1800 days, or at most 3600 days. In some embodiments, Compound A, or a pharmaceutically acceptable salt or solvate thereof is chronically administered using an amount as disclosed herein.

In some embodiments, about 280 mg/m² of Compound A, or a pharmaceutically acceptable salt or solvate thereof is chronically administered once a week over the course of 360 days. In some embodiments, about 350 mg/m² of Compound A, or a pharmaceutically acceptable salt or solvate thereof is chronically administered once a week over the course of 360 days. In some embodiments, about 420 mg/m² of Compound A, or a pharmaceutically acceptable salt or solvate thereof is chronically administered once a week over the course of 360 days. In some embodiments, about 530 mg/m² of Compound A, or a pharmaceutically acceptable salt or solvate thereof is chronically administered once a week over the course of 360 days. In some embodiments, about 280 mg/m² of Compound A, or a pharmaceutically acceptable salt or solvate thereof is chronically administered once a week for at least 1 year. In some embodiments, about 350 mg/m² of Compound A, or a pharmaceutically acceptable salt or solvate thereof is chronically administered once a week for at least 1 year. In some embodiments, about 420 mg/m² of Compound A, or a pharmaceutically acceptable salt or solvate thereof is chronically administered once a week for at least 1 year. In some embodiments, about 530 mg/m² of Compound A, or a pharmaceutically acceptable salt or solvate thereof is chronically administered once a week for at least 1 year.

In some embodiments, about 280 mg/m² of Compound A, or a pharmaceutically acceptable salt or solvate thereof is chronically administered once a week over the course of 720 days. In some embodiments, about 350 mg/m² of Compound A, or a pharmaceutically acceptable salt or solvate thereof is chronically administered once a week over the course of 720 days. In some embodiments, about 420 mg/m² of Compound A, or a pharmaceutically acceptable salt or solvate thereof is chronically administered once a week over the course of 720 days. In some embodiments, about 530 mg/m² of Compound A, or a pharmaceutically acceptable salt or solvate thereof is chronically administered once a week over the course of 720 days. In some embodiments, about 280 mg/m² of Compound A, or a pharmaceutically acceptable salt or solvate thereof is chronically administered once a week for at least 2 years. In some embodiments, about 350 mg/m² of Compound A, or a pharmaceutically acceptable salt or solvate thereof is chronically administered once a week for at least 2 years. In some embodiments, about 420 mg/m² of Compound A, or a pharmaceutically acceptable salt or solvate thereof is chronically administered once a week for at least 2 years. In some embodiments, about 400 to about 450 mg/m² of Compound A, or a pharmaceutically acceptable salt or solvate thereof is chronically administered once a week for at least 2 years. In some embodiments, about 410 and 430 mg/m² of Compound A, or a pharmaceutically acceptable salt or solvate thereof is chronically administered once a week for at least 2 years. In some embodiments, about 500 to about 550 mg/m² of Compound A, or a pharmaceutically acceptable salt or solvate thereof is chronically administered once a week for at least 2 years. In some embodiments, about 530 mg/m² of Compound A, or a pharmaceutically acceptable salt or solvate thereof is chronically administered once a week over for at least 2 years. In some embodiments, at least 530 mg/m² of Compound A, or a pharmaceutically acceptable salt or solvate thereof is chronically administered once a week for at least 2 years.

In some embodiments, Compound A or a pharmaceutically acceptable salt or solvate thereof is chronically administered in 28 day treatment cycles over a course of 30 days, 60 days, 120 days, 180 days, 240 days, 300 days, 360 days, 720 days, 1440 days, 1880 days, or 3600 days. In some embodiments, Compound A or a pharmaceutically acceptable salt or solvate thereof is chronically administered in 28 day treatment cycles over a course of at least 30 days, at least 60 days, at least 120 days, at least 180 days, at least 240 days, at least 300 days, at least 360 days, at least 720 days, at least 1440 days, at least 1880 days, or at least 3600 days. In some embodiments, Compound A or a pharmaceutically acceptable salt or solvate thereof is chronically administered in 28 day treatment cycles over a course of at most 30 days, at most 60 days, most 120 days, at most 180 days, at most 240 days, at most 300 days, at most 360 days, at most 720 days, at most 1440 days, at most 1800 days, or at most 3600. In some embodiments, Compound A, or a pharmaceutically acceptable salt or solvate thereof is chronically administered using an amount as disclosed herein.

In some embodiments, about 280 mg/m² of Compound A or a pharmaceutically acceptable salt or solvate thereof is chronically administered in 28 day treatment cycles over a course of 360 days. In some embodiments, about 350 mg/m² of Compound A or a pharmaceutically acceptable salt or solvate thereof is chronically administered in 28 day treatment cycles over a course of 360 days. In some embodiments, about 420 mg/m² of Compound A or a pharmaceutically acceptable salt or solvate thereof is chronically administered in 28 day treatment cycles over a course of 360 days. In some embodiments, about 530 mg/m² of Compound A or a pharmaceutically acceptable salt or solvate thereof is chronically administered in 28 day treatment cycles over a course of 360 days. In some embodiments, about 280 mg/m² of Compound A or a pharmaceutically acceptable salt or solvate thereof is chronically administered in 28 day treatment cycles over a course of 1 year. In some embodiments, about 350 mg/m² of Compound A or a pharmaceutically acceptable salt or solvate thereof is chronically administered in 28 day treatment cycles over a course of 1 year. In some embodiments, about 420 mg/m² of Compound A or a pharmaceutically acceptable salt or solvate thereof is chronically administered in 28 day treatment cycles over a course of 1 year. In some embodiments, about 530 mg/m² of Compound A or a pharmaceutically acceptable salt or solvate thereof is chronically administered in 28 day treatment cycles over a course of 1 year.

In some embodiments, about 280 mg/m² of Compound A or a pharmaceutically acceptable salt or solvate thereof is chronically administered in 28 day treatment cycles over a course of 720 days. In some embodiments, about 350 mg/m² of Compound A or a pharmaceutically acceptable salt or solvate thereof is chronically administered in 28 day treatment cycles over a course of 720 days. In some embodiments, about 420 mg/m² of Compound A or a pharmaceutically acceptable salt or solvate thereof is chronically administered in 28 day treatment cycles over a course of 720 days. In some embodiments, about 530 mg/m² of Compound A or a pharmaceutically acceptable salt or solvate thereof is chronically administered in 28 day treatment cycles over a course of 720 days. In some embodiments, about 280 mg/m² of Compound A or a pharmaceutically acceptable salt or solvate thereof is chronically administered in 28 day treatment cycles over a course of at least 2 years. In some embodiments, about 350 mg/m² of Compound A or a pharmaceutically acceptable salt or solvate thereof is chronically administered in 28 day treatment cycles over a course of at least 2 years. In some embodiments, about 420 mg/m² of Compound A or a pharmaceutically acceptable salt or solvate thereof is chronically administered in 28 day treatment cycles over a course of at least 2 years. In some embodiments, about 400 to about 450 mg/m² of Compound A or a pharmaceutically acceptable salt or solvate thereof is chronically administered in 28 day treatment cycles over a course of at least 2 years. In some embodiments, about 410 to about 430 mg/m² of Compound A or a pharmaceutically acceptable salt or solvate thereof is chronically administered in 28 day treatment cycles over a course of at least 2 years. In some embodiments, about 530 mg/m² of Compound A or a pharmaceutically acceptable salt or solvate thereof is chronically administered in 28 day treatment cycles over a course of at least 2 years.

In some embodiments, Compound A or a pharmaceutically acceptable salt or solvate thereof is administered up to a maximum dose once weekly (QW). In some embodiments, the maximum dosing once weekly (QW) of Compound A or a pharmaceutically acceptable salt or solvate thereof, is higher than 600 mg. In some embodiments, the maximum dosing once weekly (QW) of Compound A or a pharmaceutically acceptable salt or solvate thereof, is at most 600 mg. In some embodiments, the maximum dosing once weekly (QW) of Compound A or a pharmaceutically acceptable salt or solvate thereof, is at most 530 mg. In some embodiments, the maximum dosing once weekly (QW) of Compound A or a pharmaceutically acceptable salt or solvate thereof, is at most 420 mg. In some embodiments, the maximum dosing once weekly (QW), is at most 350 mg. In some embodiments, the maximum dosing once weekly (QW) of Compound A or a pharmaceutically acceptable salt or solvate thereof, is at most 280 mg. In some embodiments, the maximum dosing once weekly (QW) of Compound A or a pharmaceutically acceptable salt or solvate thereof, is 600 mg. In some embodiments, the maximum dosing once weekly (QW) of Compound A or a pharmaceutically acceptable salt or solvate thereof, is 530 mg. In some embodiments, the maximum dosing once weekly (QW) of Compound A or a pharmaceutically acceptable salt or solvate thereof, is 420 mg. In some embodiments, the maximum dosing once weekly (QW), is 350 mg. In some embodiments, the maximum dosing once weekly (QW) of Compound A or a pharmaceutically acceptable salt or solvate thereof, is 280 mg. In some embodiments, the Compound A or a pharmaceutically acceptable salt or solvate thereof is Compound A.

In some embodiments, Compound A or a pharmaceutically acceptable salt or solvate thereof is orally administered (PO) up to a maximum dose once weekly (QW). In some embodiments, the maximum oral dose (PO) administered once weekly (QW) of Compound A or a pharmaceutically acceptable salt or solvate thereof, is higher than 600 mg. In some embodiments, the maximum oral dose (PO) administered once weekly (QW) of Compound A or a pharmaceutically acceptable salt or solvate thereof, is at most 600 mg. In some embodiments, the maximum oral dose (PO) administered once weekly (QW) of Compound A or a pharmaceutically acceptable salt or solvate thereof, is at most 530 mg. In some embodiments, the maximum oral dose (PO) administered once weekly (QW) of Compound A or a pharmaceutically acceptable salt or solvate thereof, is at most 420 mg. In some embodiments, the maximum oral dose (PO) administered once weekly (QW), is at most 350 mg. In some embodiments, the maximum oral dose (PO) administered once weekly (QW) of Compound A or a pharmaceutically acceptable salt or solvate thereof, is at most 280 mg. In some embodiments, the maximum oral dose (PO) administered once weekly (QW) of Compound A or a pharmaceutically acceptable salt or solvate thereof, is 600 mg. In some embodiments, the maximum oral dose (PO) administered once weekly (QW) of Compound A or a pharmaceutically acceptable salt or solvate thereof, is 530 mg. In some embodiments, the maximum oral dose (PO) administered once weekly (QW) of Compound A or a pharmaceutically acceptable salt or solvate thereof, is 420 mg. In some embodiments, the maximum oral dose (PO) administered once weekly (QW) of Compound A or a pharmaceutically acceptable salt or solvate thereof, is 350 mg. In some embodiments, the maximum oral dose (PO) administered once weekly (QW) of Compound A or a pharmaceutically acceptable salt or solvate thereof, is 280 mg. In some embodiments, the Compound A or a pharmaceutically acceptable salt or solvate thereof is Compound A.

Compound A, or a pharmaceutically acceptable salt or solvate thereof can be administered to a subject in a dose escalation/de-escalation scheme. In some embodiments, the dose escalation/de-escalation scheme comprises one or more cycles of dose escalation, one or more cycles of dose de-escalation, or both. In some embodiments, the initial dose is equivalent to about 200 to about 600 mg/m² of Compound A per week. In some embodiments, the initial dose is equivalent to about 400 to about 500 mg/m² of Compound A per week (e.g., 420 mg/m² of Compound A once a week). In some embodiments, the initial dose is equivalent to about 500 to about 600 mg/m² of Compound A per week. Exemplary dose escalation/de-escalation schemes are illustrated in Tables 1-A, 1-B, and 1-C. As shown in Tables 1A to 1C, the subjects can be administered at the initial dose. If a subject does not tolerate the initial dose, a reduced dose can be administered. If the subject does not tolerate the reduced dose, a further reduced dose can be administered. If a subject tolerates the initial dose, the subject can be administered an increased dose (see Table 1-A or Table 1-C) or continue to be administered at the initial dose (see Table 1-B).

TABLE 1-A
Exemplary dose escalation/de-escalation scheme

| Dose Level | Dose of Compound A |
|---|---|
| Level −2 | about 180 mg/m² dose once weekly |
| Level −1 | about 225 mg/m² dose once weekly |
| Initial Dose | about 280 mg/m² dose once weekly |
| Level 2 | about 350 mg/m² dose once weekly |
| Level 3 | about 420 mg/m² dose once weekly |
| Level 4 | about 530 mg/m² dose once weekly |

TABLE 1-B
Exemplary dose escalation/de-escalation schemes

| Dose Level | Dose of Compound A |
|---|---|
| Level −2 | Further reduced dose |
| Level −1 | about 420 mg/m² dose once weekly |
| Initial Dose | about 530 mg/m² dose once weekly |
| | If the subject tolerates this initial dose, then |
| | Compound A will continue to be administered |
| | at the same dose |

TABLE 1-C
Exemplary dose escalation/de-escalation scheme

| | Dose of Compound A | Dose of Compound A |
|---|---|---|
| Dose Level | BSA ≤1.5 m² | BSA >1.5 m² |
| Level −1 | about 350 mg/m² dose | about 350 mg/m² dose |
| | once weekly | once weekly |
| Initial Dose | about 420 mg/m² dose | about 420 mg/m² dose |
| | once weekly | once weekly |
| Level 2 | about 530 mg/m² dose | about 530 mg/m² dose |
| | once weekly | once weekly |
| Level 3 | about 660 mg/m² dose | about 660 mg/m² dose |
| | once weekly | once weekly |
| Level 4 | about 825 mg/m² dose | about 825 mg/m² dose |
| | once weekly | once weekly |

Compound A is a Raf kinase inhibitor with a long half life which can support once weekly dosing (QW). In some embodiments, Compound A is administered once weekly with a rest period of 6 days between each administration. Suitable weekly dosages of a Raf inhibitor e.g., Compound A, or a pharmaceutically acceptable salt or solvate thereof, can generally range, in single or divided or multiple doses, up to about 1500 mg once weekly (QW). In some embodiments, compound A, or a pharmaceutically acceptable salt or solvate thereof, is administered in single or divided or multiple doses, up to 1500 mg once weekly (QW). In some embodiments, Compound A, or a pharmaceutically acceptable salt or solvate thereof, is administered up to about 1000 mg once weekly. In some embodiments, Compound A, or a pharmaceutically acceptable salt or solvate thereof, is administered up to 1000 mg once weekly. In some embodiments, Compound A, or a pharmaceutically acceptable salt or solvate thereof, is administered as a single dose. In some embodiments, Compound A, or a pharmaceutically acceptable salt or solvate thereof, is administered QW in an amount of up to 600 mg per dose. In some embodiments, Compound A, or a pharmaceutically acceptable salt or solvate thereof, is administered orally in an amount of up to 600 mg once a week. In some embodiments, Compound A, or a pharmaceutically acceptable salt or solvate thereof, is administered in an amount of up to 600 mg per dose on days 2, 9, 16, and 23 of a 28-day cycle). In some embodiments, Compound A, or a pharmaceutically acceptable salt or solvate thereof, is administered as a divided dose. In some embodiments, Compound A, or a pharmaceutically acceptable salt or solvate thereof, is administered as a divided dose on the same day. In some embodiments, Compound A, or a pharmaceutically acceptable salt or solvate thereof, is administered in multiple doses. Suitable weekly dosages include from up to about 1000 mg per dose once a week with a rest period of 6 days between each administration. In some embodiments, Compound A, or a pharmaceutically acceptable salt or solvate thereof, is administered from up to 1000 mg per dose once a week with a rest period of 6 days between each administration. Other suitable weekly dosages of Compound A, or a pharmaceutically acceptable salt or solvate thereof, can generally range, in single or divided or multiple doses from about 200 mg to about 1000 mg per dose once a week. Other suitable weekly dosages of Compound A, or a pharmaceutically acceptable salt or solvate thereof, can generally range, in single or divided or multiple doses, from about 400 mg to about 1000 mg. In some embodiment, the suitable weekly dosage is from about 400 mg to about 900 mg per dose once a week. In some embodiments, the suitable weekly dosage is from about 500 mg to about 900 mg per dose once a week. In some other embodiments, the suitable weekly dosage is from about 400 mg to about 600 mg per dose once a week. In some other embodiments, the suitable weekly dosage is from about 200 mg to about 500 mg per dose once a week. In some other embodiments, the suitable weekly dosage is from about 200 mg to about 300 mg per dose once a week. In some embodiments, suitable weekly dosages are about 200 mg, 300 mg, about 400 mg, about 500 mg, about 600 mg, about 700 mg, about 800 mg, or about 900 mg per dose once a week. In some embodiments, the QW dosing schedule differentiates the combination of Compound A, or a pharmaceutically acceptable salt or solvate thereof, and taxane based on superior safety from other available therapies. In some embodiments, the QW dosing schedule differentiates the combination of Compound A, or a pharmaceutically acceptable salt or solvate thereof, and taxane based on superior efficacy from other available therapies.

Compound A may be also administered once per week in 28 day treatment cycles. In some embodiments, the starting dose is 400, then 600, then 800 mg. In some embodiments, a maximum tolerated dose is reached at 200 mg every other day treatment schedules. In some embodiments, a maximum tolerated dose is reached at 600 mg once per week treatment schedules.

The dosage of the Raf inhibitor administered to a subject will also depend on frequency of administration. In some embodiments, Compound A, or a pharmaceutically acceptable salt or solvate thereof, is administered once weekly (QW) with a rest period of 6 days between each administration. In some embodiments, Compound A, or a pharmaceutically acceptable salt or solvate thereof, is administered daily. In some embodiments, Compound A, or a pharmaceutically acceptable salt or solvate thereof, is administered every other day. In some embodiments, Compound A, or a pharmaceutically acceptable salt or solvate thereof, is administered on a 22-day cycle in which Compound A. In some embodiments, Compound A, or a pharmaceutically acceptable salt or solvate thereof, is administered on a 28-day cycle in which Compound A. In some embodiments, Compound A, or a pharmaceutically acceptable salt or solvate thereof, is administered on a 28-day cycle in which Compound A, or a pharmaceutically acceptable salt or solvate thereof, is administered on days 1, 3, 5, 8, 10, 12, 15, 17, 19, 22, 24, and 26 of a 28-day cycle. In some embodiments, Compound A, or a pharmaceutically acceptable salt or solvate thereof, is administered on a 28-day cycle in which Compound A, or a pharmaceutically acceptable salt or solvate thereof, is administered on days 2, 9, 16, and 23 of a 28-day cycle. In some embodiments, Compound A, or a pharmaceutically acceptable salt or solvate thereof, is administered for at least 26 cycles. In some embodiments, Compound A, or a pharmaceutically acceptable salt or solvate thereof, is administered for at least 5, 6, 7, 8, 9, 10, 11, 12, 13, 14, 15, 16, 17, 18, 19, 20, 21, 22, 23, 24, 25, 26, 27, 28, 29, or 30 cycles. In some embodiments, Compound A, or a pharmaceutically acceptable salt or solvate thereof, is administered for at least 20, 21, 22, 23, 24, 25, 26, 27, 28, 29, 30, 31, 32, 33, 34, 35, 36, or 40 cycles. In some embodiments, Compound A, or a pharmaceutically acceptable salt or solvate thereof, is administered for at least 24 months, 25 months, 26 months, 27 months, 28 months, 29 months, 30 months, 31 months, 32 months, 33 months, 34 months, 35 months, 36 months, or 48 months. In some embodiments, the Compound A or a pharmaceutically acceptable salt thereof is administered to a subject for a period of at least 8 weeks. In some embodiments, the Compound A or a pharmaceutically acceptable salt thereof is administered to a subject for a period of at least 10 weeks. In some embodiments, the Compound A or a pharmaceutically acceptable salt thereof is administered to a subject for a period of at least 10.5 weeks. In some embodiments, the Compound A or a pharmaceutically acceptable salt thereof is administered to a subject for a period of at least 12 weeks. In some embodiments, the Compound A or a pharmaceutically acceptable salt thereof is administered to a subject for a period of at least 15 weeks. In some embodiments, the Compound A or a pharmaceutically acceptable salt thereof is administered to a subject for a period of at least 20 weeks. In some embodiments, the Compound A or a pharmaceutically acceptable salt thereof is administered to a subject for a period of at least 28 weeks. In some embodiments, the Compound A or a pharmaceutically acceptable salt thereof is administered to a subject for a period of at least 2 months. In some embodiments, the Compound A or a pharmaceutically acceptable salt thereof is administered to a subject for a period of at least 3 months. In some embodiments, the Compound A or a pharmaceutically acceptable salt thereof is administered to a subject for a period of at least 4 months. In some embodiments, the Compound A or a pharmaceutically acceptable salt thereof is administered to a subject for a period of at least 5 months. In some embodiments, the Compound A or a pharmaceutically acceptable salt thereof is administered to a subject for a period of at least 6 months. In some embodiments, the Compound A or a pharmaceutically acceptable salt thereof is administered to a subject for a period of at least 7 months. In some embodiments, the Compound A or a pharmaceutically acceptable salt thereof is administered to a subject for a period of at least 8 months. In some embodiments, the Compound A or a pharmaceutically acceptable salt thereof is administered to a subject for a period of at least 9 months. In some embodiments, the Compound A or a pharmaceutically acceptable salt thereof is administered to a subject for a period of at least 10 months. In some embodiments, the Compound A or a pharmaceutically acceptable salt thereof is administered to a subject for a period of at least 11 months. In some embodiments, the Compound A or a pharmaceutically acceptable salt thereof is administered to a subject for a period of at least 12 months. In some embodiments, the Compound A or a pharmaceutically acceptable salt thereof is administered to a subject for a period of at least 13 months. In some embodiments, the Compound A or a pharmaceutically acceptable salt thereof is administered to a subject for a period of at least 14 months. In some embodiments, the Compound A or a pharmaceutically acceptable salt thereof is administered to a subject for a period of at least 15 months. In some embodiments, the Compound A or a pharmaceutically acceptable salt thereof is administered to a subject for a period of at least 16 months. In some embodiments, the Compound A or a pharmaceutically acceptable salt thereof is administered to a subject for a period of at least 17 months. In some embodiments, the Compound A or a pharmaceutically acceptable salt thereof is administered to a subject for a period of at least 18 months. In some embodiments, the Compound A or a pharmaceutically acceptable salt thereof is administered to a subject for a period of at least 19 months. In some embodiments, the Compound A or a pharmaceutically acceptable salt thereof is administered to a subject for a period of at least 20 months. In some embodiments, the Compound A or a pharmaceutically acceptable salt thereof is administered to a subject for a period of at least 21 months. In some embodiments, the Compound A or a pharmaceutically acceptable salt thereof is administered to a subject for a period of at least 22 months. In some embodiments, the Compound A or a pharmaceutically acceptable salt thereof is administered to a subject for a period of at least 23 months. In some embodiments, the Compound A or a pharmaceutically acceptable salt thereof is administered to a subject for a period of at least 24 months. In some embodiments, the Compound A or a pharmaceutically acceptable salt thereof is administered to a subject for a period of at least 25 months. In some embodiments, the Compound A or a pharmaceutically acceptable salt thereof is administered to a subject for a period of at least 26 months. In some embodiments, the Compound A or a pharmaceutically acceptable salt thereof is administered to a subject for a period of at least 27 months. In some embodiments, the Compound A or a pharmaceutically acceptable salt thereof is administered to a subject for a period of at least 28 months. In some embodiments, the Compound A or a pharmaceutically acceptable salt thereof is administered to a subject for a period of at least 29 months. In some embodiments, the Compound A or a pharmaceutically acceptable salt thereof is administered to a subject for a period of at least 30 months. In some embodiments, the Compound A or a pharmaceutically acceptable salt thereof is administered to a subject for a period of at least 36 months. In some embodiments, the Compound A or a pharmaceutically acceptable salt thereof is administered to a subject for a period of at least 5 years. In some embodiments, the Compound A or a pharmaceutically acceptable salt thereof is administered to a subject for a period of at least 10 years. In some embodiments, the Compound A or a pharmaceutically acceptable salt thereof is administered to a subject chronically. In some embodiments, the Compound A or a pharmaceutically acceptable salt thereof is administered to a subject at 400 mg/m² to about 600 mg/m² (e.g., 420 mg/m²) of Compound A per week. In some embodiments, the Compound A or a pharmaceutically acceptable salt thereof is Compound A.

In one aspect, disclosed herein are methods of treating glioma (such as pediatric low grade glioma) by administering Compound A, or a pharmaceutically acceptable salt or solvate thereof to achieve a prescribed pharmacokinetic profile. During or following administration of Compound A, or a pharmaceutically acceptable salt or solvate thereof, plasma concentrations of Compound A, or a pharmaceutically acceptable salt or solvate thereof, can be determined with a validated bioanalytical assay. For example, the following pharmacokinetic (PK) parameters can be calculated where appropriate: maximum observed blood plasma concentration (Cmax), area under the concentration versus time curve from time 0 to t (AUC0-t), and apparent oral clearance of drug (CL/F).

In some embodiments, disclosed herein are methods of treating a glioma (such as pediatric low grade glioma) by administering Compound A, or a pharmaceutically acceptable salt or solvate thereof to achieve a prescribed Cmax level. When Compound A, or a pharmaceutically acceptable salt or solvate thereof, is administered, a Cmax can be measured. In some embodiments, the method comprises administering an amount of Compound A or a pharmaceutically acceptable salt or solvate thereof that is sufficient to achieve in the subject a Cmax of Compound A of at least 2000 ng/mL. In some embodiments, the method comprises administering an amount of Compound A or a pharmaceutically acceptable salt or solvate thereof that is sufficient to achieve in the subject a Cmax of Compound A of at least 2500 ng/mL, at least 3000 ng/mL, at least 3500 ng/mL, at least 4000 ng/mL, at least 4500 ng/mL, at least 5000 ng/mL, at least 5500 ng/mL, at least 6000 ng/mL, at least 6500 ng/mL, at least 7000 ng/mL, at least 7500 ng/mL, or at least 8000 ng/mL.

In some embodiments, the described method comprises administering an amount of Compound A or a pharmaceutically acceptable salt or solvate thereof that is sufficient to achieve in the subject a Cmax of Compound A that is within a suitable range. In some embodiments, the Cmax is about 2,000 ng/mL to about 8,000 ng/mL. In some embodiments, the Cmax is at least about 2,000 ng/mL. In some embodiments, the Cmax is at most about 8,000 ng/mL. In some embodiments, the method comprises administering an amount of Compound A or a pharmaceutically acceptable salt or solvate thereof that is sufficient to achieve in the subject a Cmax of Compound A that is about 2,000 ng/mL to about 2,500 ng/mL, about 2,000 ng/mL to about 3,000 ng/mL, about 2,000 ng/mL to about 3,500 ng/mL, about 2,000 ng/mL to about 4,000 ng/mL, about 2,000 ng/mL to about 4,500 ng/mL, about 2,000 ng/mL to about 5,000 ng/mL, about 2,000 ng/mL to about 5,500 ng/mL, about 2,000 ng/mL to about 6,000 ng/mL, about 2,000 ng/mL to about 6,500 ng/mL, about 2,000 ng/mL to about 7,000 ng/mL, about 2,000 ng/mL to about 8,000 ng/mL, about 2,500 ng/mL to about 3,000 ng/mL, about 2,500 ng/mL to about 3,500 ng/mL, about 2,500 ng/mL to about 4,000 ng/mL, about 2,500 ng/mL to about 4,500 ng/mL, about 2,500 ng/mL to about 5,000 ng/mL, about 2,500 ng/mL to about 5,500 ng/mL, about 2,500 ng/mL to about 6,000 ng/mL, about 2,500 ng/mL to about 6,500 ng/mL, about 2,500 ng/mL to about 7,000 ng/mL, about 2,500 ng/mL to about 8,000 ng/mL, about 3,000 ng/mL to about 3,500 ng/mL, about 3,000 ng/mL to about 4,000 ng/mL, about 3,000 ng/mL to about 4,500 ng/mL, about 3,000 ng/mL to about 5,000 ng/mL, about 3,000 ng/mL to about 5,500 ng/mL, about 3,000 ng/mL to about 6,000 ng/mL, about 3,000 ng/mL to about 6,500 ng/mL, about 3,000 ng/mL to about 7,000 ng/mL, about 3,000 ng/mL to about 8,000 ng/mL, about 3,500 ng/mL to about 4,000 ng/mL, about 3,500 ng/mL to about 4,500 ng/mL, about 3,500 ng/mL to about 5,000 ng/mL, about 3,500 ng/mL to about 5,500 ng/mL, about 3,500 ng/mL to about 6,000 ng/mL, about 3,500 ng/mL to about 6,500 ng/mL, about 3,500 ng/mL to about 7,000 ng/mL, about 3,500 ng/mL to about 8,000 ng/mL, about 4,000 ng/mL to about 4,500 ng/mL, about 4,000 ng/mL to about 5,000 ng/mL, about 4,000 ng/mL to about 5,500 ng/mL, about 4,000 ng/mL to about 6,000 ng/mL, about 4,000 ng/mL to about 6,500 ng/mL, about 4,000 ng/mL to about 7,000 ng/mL, about 4,000 ng/mL to about 8,000 ng/mL, about 4,500 ng/mL to about 5,000 ng/mL, about 4,500 ng/mL to about 5,500 ng/mL, about 4,500 ng/mL to about 6,000 ng/mL, about 4,500 ng/mL to about 6,500 ng/mL, about 4,500 ng/mL to about 7,000 ng/mL, about 4,500 ng/mL to about 8,000 ng/mL, about 5,000 ng/mL to about 5,500 ng/mL, about 5,000 ng/mL to about 6,000 ng/mL, about 5,000 ng/mL to about 6,500 ng/mL, about 5,000 ng/mL to about 7,000 ng/mL, about 5,000 ng/mL to about 8,000 ng/mL, about 5,500 ng/mL to about 6,000 ng/mL, about 5,500 ng/mL to about 6,500 ng/mL, about 5,500 ng/mL to about 7,000 ng/mL, about 5,500 ng/mL to about 8,000 ng/mL, about 6,000 ng/mL to about 6,500 ng/mL, about 6,000 ng/mL to about 7,000 ng/mL, about 6,000 ng/mL to about 8,000 ng/mL, about 6,500 ng/mL to about 7,000 ng/mL, about 6,500 ng/mL to about 8,000 ng/mL, or about 7,000 ng/mL to about 8,000 ng/mL.

In some embodiments, disclosed herein are methods of treating a glioma (such as pediatric low grade glioma) by administering Compound A, or a pharmaceutically acceptable salt or solvate thereof to achieve a prescribed AUC level of Compound A. In some embodiments, the area under the concentration versus time curve from time 0 to t (AUC0-t) or AUCₛₛ (steady-state AUC) is measured in a subject administered Compound A, or a pharmaceutically acceptable salt or solvate thereof. In some embodiments, AUC0-t is AUC0-12 (or AUC0-12 hr), AUC0-24 (or AUC0-24 hr), or AUC 0-48 (or AUC0-48 hr). In some embodiments, the AUC0-t is AUC0-24. In some embodiments, the AUC is AUCₛₛ.

In some embodiments, a method described herein comprises administering an amount of Compound A or a pharmaceutically acceptable salt or solvate thereof that is sufficient to achieve in the subject a prescribed steady-state AUC (AUCₛₛ). In some embodiments, the method comprises administering an amount of Compound A or a pharmaceutically acceptable salt or solvate thereof that is sufficient to achieve in the subject an AUCₛₛ for Compound A that is at least about 100,000 ng*h/mL. In some embodiments, the AUCₛₛ is at least about 200,000 ng*h/mL. In some embodiments, the AUCₛₛ is at least about 300,000 ng*h/mL. In some embodiments, the method comprises administering an amount of Compound A or a pharmaceutically acceptable salt or solvate thereof that is sufficient to achieve in the subject an AUCₛₛ for Compound A that is at least about 400,000 ng*h/mL. In some embodiments, the AUCₛₛ is at least about 500,000 ng*h/mL. In some embodiments, the AUCₛₛ is at least about 600,000 ng*h/mL. In some embodiments, the AUCₛₛ is at least about 400,000 ng*h/mL to at least about 800,000 ng*h/mL. In some embodiments, the AUCₛₛ is at least about 500,000 ng*h/mL to at least about 700,000 ng*h/mL. In some embodiments, the AUCₛₛ is at least about 300,000 ng*h/mL to at least about 800,000 ng*h/mL. In some embodiments, the AUCₛₛ is at least about 200,000 ng*h/mL to at least about 800,000 ng*h/mL. In some embodiments, the AUCₛₛ is about 100,000 ng*h/mL to about 800,000 ng*h/mL. In some embodiments, the AUCₛₛ is at most about 600,000 ng*h/mL. In some embodiments, the AUCₛₛ is at most about 800,000 ng*h/mL. In some embodiments, the AUCₛₛ is at most about 1,000,000 ng*h/mL. In some embodiments, the AUCₛₛ is at most about 1,200,000 ng*h/mL. In some embodiments, the AUCₛₛ is at most about 1,600,000 ng*h/mL. In some embodiments, the method comprises administering an amount of Compound A or a pharmaceutically acceptable salt or solvate thereof that is sufficient to achieve in the subject an AUCₛₛ of Compound A that is about 100,000 ng*h/mL to about 1,600,000 ng*h/mL, about 100,000 ng*h/mL to about 1,000,000 ng*h/mL, about 100,000 ng*h/mL to about 800,000 ng*h/mL, about 100,000 ng*h/mL to about 600,000 ng*h/mL, 200,000 ng*h/mL to about 1,600,000 ng*h/mL, about 200,000 ng*h/mL to about 1,000,000 ng*h/mL, about 200,000 ng*h/mL to about 800,000 ng*h/mL, about 200,000 ng*h/mL to about 600,000 ng*h/mL, 300,000 ng*h/mL to about 1,600,000 ng*h/mL, about 300,000 ng*h/mL to about 1,000,000 ng*h/mL, about 300,000 ng*h/mL to about 800,000 ng*h/mL, about 300,000 ng*h/mL to about 600,000 ng*h/mL, 400,000 ng*h/mL to about 1,600,000 ng*h/mL, about 400,000 ng*h/mL to about 1,000,000 ng*h/mL, about 400,000 ng*h/mL to about 800,000 ng*h/mL, about 400,000 ng*h/mL to about 600,000 ng*h/mL, 500,000 ng*h/mL to about 1,600,000 ng*h/mL, about 500,000 ng*h/mL to about 1,000,000 ng*h/mL, about 500,000 ng*h/mL to about 800,000 ng*h/mL, about 500,000 ng*h/mL to about 600,000 ng*h/mL, 600,000 ng*h/mL to about 1,600,000 ng*h/mL, about 600,000 ng*h/mL to about 1,000,000 ng*h/mL, or about 600,000 ng*h/mL to about 800,000 ng*h/mL. In some embodiments, the method comprises administering an amount of Compound A or a pharmaceutically acceptable salt or solvate thereof that is sufficient to achieve in the subject an AUCₛₛ of Compound A that is about 300,000 ng*h/mL to about 450,000 ng*h/mL, about 300,000 ng*h/mL to about 500,000 ng*h/mL, about 300,000 ng*h/mL to about 550,000 ng*h/mL, about 300,000 ng*h/mL to about 650,000 ng*h/mL, about 350,000 ng*h/mL to about 750,000 ng*h/mL, about 400,000 ng*h/mL to about 650,000 ng*h/mL, about 400,000 ng*h/mL to about 750,000 ng*h/mL, about 400,000 ng*h/mL to about 850,000 ng*h/mL, about 400,000 ng*h/mL to about 950,000 ng*h/mL, or about 400,000 ng*h/mL to about 1,000,000 ng*h/mL.

In some embodiments, a method described herein comprises administering an amount of Compound A or a pharmaceutically acceptable salt or solvate thereof that is sufficient to achieve in the subject a prescribed AUC04. In some embodiments, the AUC0-t is AUC0-24. In some embodiments, the method comprises administering an amount of Compound A or a pharmaceutically acceptable salt or solvate thereof that is sufficient to achieve in the subject an AUC0-24 for Compound A that is at least about 10,000 ng*h/mL. In some embodiments, the method comprises administering an amount of Compound A or a pharmaceutically acceptable salt or solvate thereof that is sufficient to achieve in the subject an AUC0-24 of Compound A that is at least about 50,000 ng*h/mL. In some embodiments, the method comprises administering an amount of Compound A or a pharmaceutically acceptable salt or solvate thereof that is sufficient to achieve in the subject an AUC0-24 of Compound A that is at least about 100,000 ng*h/mL. In some embodiments, the AUC0-24 is at least about 100,000 ng*h/mL to at least about 600,000 ng*h/mL. In some embodiments, the AUC0-24 is about 100,000 ng*h/mL to about 600,000 ng*h/mL. In some embodiments, the AUC0-24 is at least about 100,000 ng*h/mL. In some embodiments, the AUC0-24 is at most about 600,000 ng*h/mL. In some embodiments, the method comprises administering an amount of Compound A or a pharmaceutically acceptable salt or solvate thereof that is sufficient to achieve in the subject an AUC0-24 of Compound A that is about 100,000 ng*h/mL to about 150,000 ng*h/mL, about 100,000 ng*h/mL to about 200,000 ng*h/mL, about 100,000 ng*h/mL to about 250,000 ng*h/mL, about 100,000 ng*h/mL to about 300,000 ng*h/mL, about 100,000 ng*h/mL to about 350,000 ng*h/mL, about 100,000 ng*h/mL to about 400,000 ng*h/mL, about 100,000 ng*h/mL to about 450,000 ng*h/mL, about 100,000 ng*h/mL to about 500,000 ng*h/mL, about 100,000 ng*h/mL to about 550,000 ng*h/mL, about 100,000 ng*h/mL to about 600,000 ng*h/mL, about 150,000 ng*h/mL to about 200,000 ng*h/mL, about 150,000 ng*h/mL to about 250,000 ng*h/mL, about 150,000 ng*h/mL to about 300,000 ng*h/mL, about 150,000 ng*h/mL to about 350,000 ng*h/mL, about 150,000 ng*h/mL to about 400,000 ng*h/mL, about 150,000 ng*h/mL to about 450,000 ng*h/mL, about 150,000 ng*h/mL to about 500,000 ng*h/mL, about 150,000 ng*h/mL to about 550,000 ng*h/mL, about 150,000 ng*h/mL to about 600,000 ng*h/mL, about 200,000 ng*h/mL to about 250,000 ng*h/mL, about 200,000 ng*h/mL to about 300,000 ng*h/mL, about 200,000 ng*h/mL to about 350,000 ng*h/mL, about 200,000 ng*h/mL to about 400,000 ng*h/mL, about 200,000 ng*h/mL to about 450,000 ng*h/mL, about 200,000 ng*h/mL to about 500,000 ng*h/mL, about 200,000 ng*h/mL to about 550,000 ng*h/mL, about 200,000 ng*h/mL to about 600,000 ng*h/mL, about 250,000 ng*h/mL to about 300,000 ng*h/mL, about 250,000 ng*h/mL to about 350,000 ng*h/mL, about 250,000 ng*h/mL to about 400,000 ng*h/mL, about 250,000 ng*h/mL to about 450,000 ng*h/mL, about 250,000 ng*h/mL to about 500,000 ng*h/mL, about 250,000 ng*h/mL to about 550,000 ng*h/mL, about 250,000 ng*h/mL to about 600,000 ng*h/mL, about 300,000 ng*h/mL to about 350,000 ng*h/mL, about 300,000 ng*h/mL to about 400,000 ng*h/mL, about 300,000 ng*h/mL to about 450,000 ng*h/mL, about 300,000 ng*h/mL to about 500,000 ng*h/mL, about 300,000 ng*h/mL to about 550,000 ng*h/mL, about 300,000 ng*h/mL to about 600,000 ng*h/mL, about 350,000 ng*h/mL to about 400,000 ng*h/mL, about 350,000 ng*h/mL to about 450,000 ng*h/mL, about 350,000 ng*h/mL to about 500,000 ng*h/mL, about 350,000 ng*h/mL to about 550,000 ng*h/mL, about 350,000 ng*h/mL to about 600,000 ng*h/mL, about 400,000 ng*h/mL to about 450,000 ng*h/mL, about 400,000 ng*h/mL to about 500,000 ng*h/mL, about 400,000 ng*h/mL to about 550,000 ng*h/mL, about 400,000 ng*h/mL to about 600,000 ng*h/mL, about 450,000 ng*h/mL to about 500,000 ng*h/mL, about 450,000 ng*h/mL to about 550,000 ng*h/mL, about 450,000 ng*h/mL to about 600,000 ng*h/mL, about 500,000 ng*h/mL to about 550,000 ng*h/mL, about 500,000 ng*h/mL to about 600,000 ng*h/mL, or about 550,000 ng*h/mL to about 600,000 ng*h/mL.

In some embodiments, a method described herein comprises administering an amount of Compound A or a pharmaceutically acceptable salt or solvate thereof that is sufficient to achieve in the subject a prescribed AUC₀₋₂₈. In some embodiments, the AUC0-∞ of Compound A comprises about 250 μg·hr/L to about 1,600 μg·hr/L. In some embodiments, the AUC0-∞ of Compound A comprises at least about 250 μg·hr/L. In some embodiments, the AUC0-∞ of Compound A comprises at most about 1,600 μg·hr/L. In some embodiments, the AUC0-∞ of Compound A comprises about 250 μg·hr/L to about 350 μg·hr/L, about 250 μg·hr/L to about 450 μg·hr/L, about 250 μg·hr/L to about 550 μg·hr/L, about 250 μg·hr/L to about 650 μg·hr/L, about 250 μg·hr/L to about 750 μg·hr/L, about 250 μg·hr/L to about 850 μg·hr/L, about 250 μg·hr/L to about 950 μg·hr/L, about 250 μg·hr/L to about 1,000 μg·hr/L, about 250 μg·hr/L to about 1,250 μg·hr/L, about 250 μg·hr/L to about 1,500 μg·hr/L, about 250 μg·hr/L to about 1,600 μg·hr/L, about 350 μg·hr/L to about 450 μg·hr/L, about 350 μg·hr/L to about 550 μg·hr/L, about 350 μg·hr/L to about 650 μg·hr/L, about 350 μg·hr/L to about 750 μg·hr/L, about 350 μg·hr/L to about 850 μg·hr/L, about 350 μg·hr/L to about 950 μg·hr/L, about 350 μg·hr/L to about 1,000 μg·hr/L, about 350 μg·hr/L to about 1,250 μg·hr/L, about 350 μg·hr/L to about 1,500 μg·hr/L, about 350 μg·hr/L to about 1,600 μg·hr/L, about 450 μg·hr/L to about 550 μg·hr/L, about 450 μg·hr/L to about 650 μg·hr/L, about 450 μg·hr/L to about 750 μg·hr/L, about 450 μg·hr/L to about 850 μg·hr/L, about 450 μg·hr/L to about 950 μg·hr/L, about 450 μg·hr/L to about 1,000 μg·hr/L, about 450 μg·hr/L to about 1,250 μg·hr/L, about 450 μg·hr/L to about 1,500 μg·hr/L, about 450 μg·hr/L to about 1,600 μg·hr/L, about 550 μg·hr/L to about 650 μg·hr/L, about 550 μg·hr/L to about 750 μg·hr/L, about 550 μg·hr/L to about 850 μg·hr/L, about 550 μg·hr/L to about 950 μg·hr/L, about 550 μg·hr/L to about 1,000 μg·hr/L, about 550 μg·hr/L to about 1,250 μg·hr/L, about 550 μg·hr/L to about 1,500 μg·hr/L, about 550 μg·hr/L to about 1,600 μg·hr/L, about 650 μg·hr/L to about 750 μg·hr/L, about 650 μg·hr/L to about 850 μg·hr/L, about 650 μg·hr/L to about 950 μg·hr/L, about 650 μg·hr/L to about 1,000 μg·hr/L, about 650 μg·hr/L to about 1,250 μg·hr/L, about 650 μg·hr/L to about 1,500 μg·hr/L, about 650 μg·hr/L to about 1,600 μg·hr/L, about 750 μg·hr/L to about 850 μg·hr/L, about 750 μg·hr/L to about 950 μg·hr/L, about 750 μg·hr/L to about 1,000 μg·hr/L, about 750 μg·hr/L to about 1,250 μg·hr/L, about 750 μg·hr/L to about 1,500 μg·hr/L, about 750 μg·hr/L to about 1,600 μg·hr/L, about 850 μg·hr/L to about 950 μg·hr/L, about 850 μg·hr/L to about 1,000 μg·hr/L, about 850 μg·hr/L to about 1,250 μg·hr/L, about 850 μg·hr/L to about 1,500 μg·hr/L, about 850 μg·hr/L to about 1,600 μg·hr/L, about 950 μg·hr/L to about 1,000 μg·hr/L, about 950 μg·hr/L to about 1,250 μg·hr/L, about 950 μg·hr/L to about 1,500 μg·hr/L, about 950 μg·hr/L to about 1,600 μg·hr/L, about 1,000 μg·hr/L to about 1,250 μg·hr/L, about 1,000 μg·hr/L to about 1,500 μg·hr/L, about 1,000 μg·hr/L to about 1,600 μg·hr/L, about 1,250 μg·hr/L to about 1,500 μg·hr/L, about 1,250 μg·hr/L to about 1,600 μg·hr/L, or about 1,500 μg·hr/L to about 1,600 μg·hr/L.

### Administration of a Second Agent

In some embodiments, Compound A, or a pharmaceutically acceptable salt or solvate thereof, or a pharmaceutically acceptable salt or solvate is administered with a second therapeutic agent. In some embodiments, the second therapeutic agent is administered to treat a skin-related condition or disorder such as a acneiform rash, maculopapular rash, dry skin, or an HFSR (Rash, hand-foot skin reaction). In some embodiments, treatment of a low-grade glioma is administered with 1) Compound A and 2) one or more therapeutic agents for treating a skin-related condition or disorder. In some embodiments, the second therapeutic agent is an agent for treating one or more of a follicular reaction, an eczematous reaction, a paronychia, and a hand foot syndrome. In some embodiments, the second therapeutic agent is ketoconazole, steroid cream/ointment, topical clindamycin, oral antibiotics, and topical keratolytic.

### Subject

In some embodiments, a subject has a body surface area (BSA) of from about 0.5 m² to about 2.0 m². In some embodiments, the subject has a BSA of from about 0.5 m² to about 1.5 m². In some embodiments, the subject has a BSA of about 0.5 m², 0.75 m², 1.0 m², 1.25 m², 1.5 m², or 1.75 m². In some embodiments, the subject has a BSA of about 0.5 m² to about 1.5 m². In some embodiments, the subject has a BSA of at least about 0.5 m². In some embodiments, the subject has a BSA of at most about 2.0 m². In some embodiments, the subject has a BSA of at most about 1.9 m². In some embodiments, the subject has a BSA of at most about 1.8 m². In some embodiments, the subject has a BSA of at most about 1.7 m². In some embodiments, the subject has a BSA of at most about 1.6 m². In some embodiments, the subject has a BSA of at most about 1.5 m². In some embodiments, the subject has a BSA of at most about 1.4 m². In some embodiments, the subject has a BSA of at most about 1.3 m². In some embodiments, the subject has a BSA of at most about 1.2 m². In some embodiments, the subject has a BSA of at most about 1.1 m². In some embodiments, the subject has a BSA of at most about 1.0 m². In some embodiments, the subject has a BSA of at most about 0.9 m². In some embodiments, the subject has a BSA of at most about 0.8 m². In some embodiments, the subject has a BSA of at most about 0.7 m². In some embodiments, the subject has a BSA of at most about 0.6 m². In some embodiments, the subject has a BSA of at most about 0.5 m². In some embodiments, the subject has a BSA of at least about 0.4 m². In some embodiments, the subject has a BSA of at least about 0.5 m². In some embodiments, the subject has a BSA of at least about 0.6 m². In some embodiments, the subject has a BSA of at least about 0.7 m². In some embodiments, the subject has a BSA of at least about 0.8 m². In some embodiments, the subject has a BSA of at least about 0.9 m². In some embodiments, the subject has a BSA of at least about 1.0 m². In some embodiments, the subject has a BSA of at least about 1.1 m². In some embodiments, the subject has a BSA of at least about 1.2 m². In some embodiments, the subject has a BSA of at least about 1.3 m². In some embodiments, the subject has a BSA of at least about 1.4 m². In some embodiments, the subject has a BSA of at least about 1.5 m². In some embodiments, the subject has a BSA of about 0.5 m² to about 0.75 m², about 0.5 m² to about 1 m², about 0.5 m² to about 1.25 m², about 0.5 m² to about 1.5 m², about 0.75 m² to about 1 m², about 0.75 m² to about 1.25 m², about 0.75 m² to about 1.5 m², about 1 m² to about 1.25 m², about 1 m² to about 1.5 m², or about 1.25 m² to about 1.5 m². In some embodiments, the subject has a BSA of about 1.5 m² to about 2.0 m². In some embodiments, the subject has a BSA of about 2 m² to about 2.5 m². In some embodiments, the subject has a BSA of at least 1.5 m².

In one aspect, described herein are methods of treating low-grade gliomas (LGG) in a subject in need thereof, the method comprising administering Compound A, or a pharmaceutically acceptable salt or solvate thereof to the subject. In some embodiments, the LGG has one or more of the following mutations: RAS positive mutation, RAF positive mutation, MEK positive mutation, and ERK positive mutation. In embodiments, patients were included to have advance metastatic or respectable melanoma with MAPK mutations. In some embodiments, the LGG has a BRAF mutation. In some embodiments, the LGG has a V600E mutation. In some embodiments, the LGG has a V600D mutation. In some embodiments, the LGG has a V600K mutation. In some embodiments, the LGG has a non V600E mutation. In some embodiments, the BRAF mutation is a non V600 BRAF mutation. In some embodiments, the subject is identified having one or more of the following wild-type fusions: KIAA1549:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF21:BRAF, FXR1:BRAF, RNF130:BRAF, BRAF:MACF1, TMEM106B:BRAF, PPC1CC:BRAF, CUX1:BRAF, SRGAP3:RAF1, QK1:RAF1, FYCO:RAF1, ATG7:RAF1, and NFIA:RAF1. In some embodiments, the subject is identified having a SRGAP3:RAF1 fusion. In some embodiments, the subject is identified having KIAA1549: BRAF fusion. In some embodiments, the subject has a KIAA1549: BRAF fusion. In some embodiments, the subject has a STARD3NL:BRAF fusion. In some embodiments, the subject has a BCAS1:BRAF fusion. In some embodiments, the subject has a KHDRBS2:BRAF fusion. In some embodiments, the subject has a CCDC6:BRAF fusion. In some embodiments, the subject has a FAM131B:BRAF fusion. In some embodiments, the subject has a SRGAP:BRAF fusion. In some embodiments, the subject has a CLCN6:BRAF fusion. In some embodiments, the subject has a GNAI1:BRAF fusion. In some embodiments, the subject has a MRKN1:BRAF fusion. In some embodiments, the subject has a GIT2:BRAF fusion. In some embodiments, the subject has a GTF21:BRAF fusion. In some embodiments, the subject has a FXR1:BRAF fusion. In some embodiments, the subject has a RNF130:BRAF fusion. In some embodiments, the subject has a GTF21:BRAF fusion. In some embodiments, the subject has a BRAF:MACF1 fusion. In some embodiments, the subject has a TMEM106B:BRAF fusion. In some embodiments, the subject has a PPC1CC:BRAF fusion. In some embodiments, the subject has a CUX1:BRAF fusion. In some embodiments, the subject has a SRGAP3:RAF1 fusion. In some embodiments, the subject has a QK1:RAF1 fusion. In some embodiments, the subject has a FYCO:RAF1 fusion. In some embodiments, the subject has a ATG7:RAF1 fusion. In some embodiments, the subject has a NFIA:RAF1 fusion. In some embodiments, the subject has a BRAF gene fusion. In some embodiments, the subject has a CRAF gene fusion.

Criteria for the inclusion of a subject during the administration of Compound A may be required. In some embodiments, a subject has radiographically recurrent or radiographically progressive non hematological malignancies. In some embodiments, the hematological malignancies are derived from the CNS or solid tumors. In some embodiments, the hematological malignancies are associated with the activation of RAS, RAF<EK, ERK. In some embodiments, the subject has not been identified with NF1. In some embodiments, the subject has received at least one line of systemic therapy. In some embodiments, the subject has evidence of radiographic progression. In some embodiments, the subject has received at least one line of systemic therapy (e.g. chemotherapy) and evidence of radiographic progression. In some embodiments, the subject has relapsed LGG. In some embodiments, the subject has refractory LGG. In some embodiments, the subject has previously received a surgery for treating the LGG. In some embodiments, the subject has previously received a complete and partial surgical resection.

Criteria for secondary outcomes measure during the administration of Compound A may be considered. In some embodiments, the secondary outcome measures can include safety, pharmacokinetics, motor function, effect on ECG measurements, or visual acuity.

In some embodiments, the subject is from about 6 months to 25 years old. In some embodiments, the subject is a child. In some embodiments, the subject is an adolescent. In some embodiments, the subject is an adult. In some embodiments, the subject is from about 1 year to 25 years old. In some embodiments, a subject is 25 years of age of less. In some embodiments, a subject is 20 years of age or less. In some embodiments, a subject is 15 years of age or less. In some embodiments, a subject is 10 years of age or less. In some embodiments, a subject is 6 months to 5 years of age. In some embodiments, a subject is 6 months to 10 years of age. In some embodiments, a subject is 6 months to 15 years of age. In some embodiments, a subject is 20, 19, 18, 17, 16, 15, 14, 13, 12, 11, 10 years of age or less. In some embodiments, the subject is 1, 2, 3, 4, 5, 6, 7, 8, 9, 10, 11, 12, 13, 14, 15, 16, 17, 18, 19, 20, 21, 22, 23, 24, 25 years old. In some embodiments, the subject is measured for a performance status. In some embodiments, the performance status if the Karnofsky or Lansky performance status. In some embodiments, the Karnofsky status is greater than or equal to 50. In some embodiments, the Lansky status is greater than or equal to 50. In some embodiments, the subject has low grade glioma. In some embodiments, the subject has failed standard therapy.

### EXAMPLES

### Example 1: Compound A Treatment Schedule

Compound A will be administered at the dose of 530 mg/m² (not to exceed 800 mg) PO once weekly (QW) (Days 1, 8, 15, 22 of a 28-day cycle.). Body surface area (BSA) will be determined by the Mosteller Formula (√((height×weight)/3600)). Patients enrolled to this study will be evaluated for safety and Compound A tolerability following the first 28 days of Compound A treatment, with the Safety Review Committee (SRC) evaluating the data based on protocol-specified traditional dose limiting toxicity (DLT) criteria. If the patient tolerates the dose of 530 mg/m² per week, then Compound A will continue to be administered at the same dose. If the patient does not tolerates the dose of 530 mg/m² per week, then a reduced dose, such as 420 mg/m² will be administered. If the patient does not tolerates the dose of 420 mg/m² per week, then a further reduced dose will be administered.

Treatment cycles will repeat every 28 days in the absence of disease progression or unacceptable toxicity. Patients will undergo radiographic evaluation of their disease at the end of every third cycle, starting with the end of cycle 3 (C3). Patients will continue on Compound A until radiographic evidence of disease progression by RANO criteria or as recommended by the patient's care team for other reasons.

Patients who have radiographic evidence of disease progression may be allowed to continue Compound A if, in the opinion of the care team, the patient is deriving clinical benefit from continuing treatment. Disease assessments should continue for patients being treated beyond progression.

Compound A is administered as an oral tablet or age-appropriate formulation (suspension or sprinkle) at 530 mg/m² (not to exceed 800 mg). Patients who are able to swallow tablets will receive the tablet formulation. Approximately 50 patients will be accrued to receive the tablet formulation. Patients who are unable or unwilling to swallow tablets will receive the age-appropriate formulation, once available. Approximately 10 patients will be accrued to receive the age-appropriate formulation.

Patients will initiate treatment at 530 mg/m² QW (not to exceed 800 mg QW) cycle 1 day 1 (i.e., C1D1). Each cycle will consist of 28 days of continuous dosing.

### Example 2. Patient-Level Dose, Exposure, and Preliminary Response Data

A 3+3 design is used. Based on the adult dose of 350 mg/m² by mouth every week (using a typical adult BSA of 1.7 m²), the starting dose for the pediatric study was 80% of the adult RP2D, which was 280 mg/m² by mouth every week. Doses were escalated in an inter-patient stepwise fashion until dose-limiting toxicities (DLTs) were observed. The three different dose levels: 280 mg/m², 350 mg/m², and 420 mg/m².

Treatment began at 80% of the adult dose (i.e., 280 mg/m² by mouth once weekly based on the following calculation:
An adult dose of 600 mg once weekly and an average adult BSA (m²) of 1.73 m²=350 mg/m²
80% of 350 mg/m²=280 mg/m² starting dose

The dose escalation scheme is described in Table 1-A. The initial phase included 3 dose escalation levels to a maximum of 530 mg/m²/dose weekly.

Pharmacokinetic studies were performed on all patients in the phase I component of the trial as venous access allowed. The initial phase 1 included 3 dose escalation levels to a maximum of 530 mg/m²/dose weekly. PK studies were also conducted to evaluate phosphorylated ERK in the peripheral blood mononuclear cells.

Preliminary analysis suggested there could be a difference in response based on exposure. PK analysis of the pediatric values in mg/kg suggested an almost 2.2 fold difference. Moreover, preliminary observations noted a more robust response among patients with the lowest BSA and highest exposure.

The patient-level dose, exposure, and predicted exposure are illustrated in Table 3. The response data is illustrated in Table 2.

TABLE 2
Patient-Level Dose, Exposure, and Response Data

| | | | | Dose | Dose | | | Best RANO |
|---|---|---|---|---|---|---|---|---|
| | Age | Weight | BSA | Cohort | Given | Dose in | AUC_{0-∞} | Response |
| Patient Number | (y) | (kg) | (m²) | (mg/m²) | (mg) | mg/kg | (μg · h/L) | (T1-Gad) |
| Subject-1 | 9 | 21.9 | 0.86 | 280 | 240 | 11.0 | 670 | CR |
| (PNOC014-01) | | | | | | | | |
| Subject-2 | 12 | 28.2 | 1.0 | 280 | 280 | 9.9 | 361 | PR |
| (PNOC014-02) | | | | | | | | |
| Subject-3 | 5 | 18.1 | 0.79 | 280 | 220 | 12.2 | 324 | PR |
| (PNOC014-03) | | | | | | | | |
| Subject-4 | 12 | 69.6 | 1.71 | 350 | 600 | 8.6 | 466 | PD |
| (PNOC014-04) | | | | | | | | |
| Subject-5 | 12 | 66.5 | 1.71 | 350 | 600 | 9.0 | 431 | SD |
| (PNOC014-05) | | | | | | | | |
| Subject-6 | 14 | 53.6 | 1.6 | 350 | 560 | 10.4 | 423 | PD |
| (PNOC014-06) | | | | | | | | |
| Subject-7 | 13 | 53.7 | 1.48 | 420 | 620 | 11.5 | 556 | SD |
| (PNOC014-07) | | | | | | | | |
| Subject-8 | — | — | — | 420 | — | — | — | CR |
| (PNOC014-08) | | | | | | | | |
| Subject-9 | 4 | 15.6 | 0.71 | 420 | 300 | 19.2 | 889 | PR |
| (PNOC014-09) | | | | | | | | |

Abbreviations: AUC_{0-∞} = area under the concentration-time curve from time 0 to infinity;
BSA = body surface area;
RANO Responses (SPD): CR = −100%;
PR = −50% or more reduction from baseline;
PD = ≥25% growth from nadir,
SD = between −50% and +25%.
Subject-8 (PNOC014-08), dosed at 420 mg/m², did not have PK data, but achieved a best response of CR by RANO criteria

TABLE 3
Patient-Level Dose, Exposure, and Predicted Exposure

| | | Original | | | | Dose | Prior |
|---|---|---|---|---|---|---|---|
| | | Dose | | Predicted | Predicted | normalized | Observed |
| | Age | Cohort | BSA | dose (530 | dose | AUC_{0-∞} | AUC_{0-∞} |
| Patient Number | (y) | (mg/m²) | (m²) | mg/m²) | (mg/kg) | (μg · hr/L) | (μg · hr/L) |
| Subject-1 | 9 | 280 | 0.86 | 456 | 21 | 1268 | 670 |
| (PNOC014-01) | | | | | | | |
| Subject-2 | 12 | 280 | 1 | 530 | 19 | 682 | 361 |
| (PNOC014-02) | | | | | | | |
| Subject-3 | 5 | 280 | 0.79 | 419 | 23 | 614 | 324 |
| (PNOC014-03) | | | | | | | |
| Subject-4 | 12 | 350 | 1.71 | 906 | 13 | 705 | 466 |
| (PNOC014-04) | | | | | | | |
| Subject-5 | 12 | 350 | 1.71 | 906 | 14 | 653 | 431 |
| (PNOC014-05) | | | | | | | |
| Subject-6 | 14 | 350 | 1.6 | 848 | 16 | 640 | 423 |
| (PNOC014-06) | | | | | | | |
| Subject-7 | 13 | 420 | 1.48 | 784 | 15 | 701 | 556 |
| (PNOC014-07) | | | | | | | |
| Subject-8 | | 420 | | | | | |
| (PNOC014-08) | | | | | | | |
| Subject-9 | 4 | 420 | 0.71 | 376 | 24 | 1122 | 889 |
| (PNOC014-09) | | | | | | | |

Abbreviations: AUC_{0-∞} = area under the concentration-time curve from time 0 to infinity;
BSA = body surface area

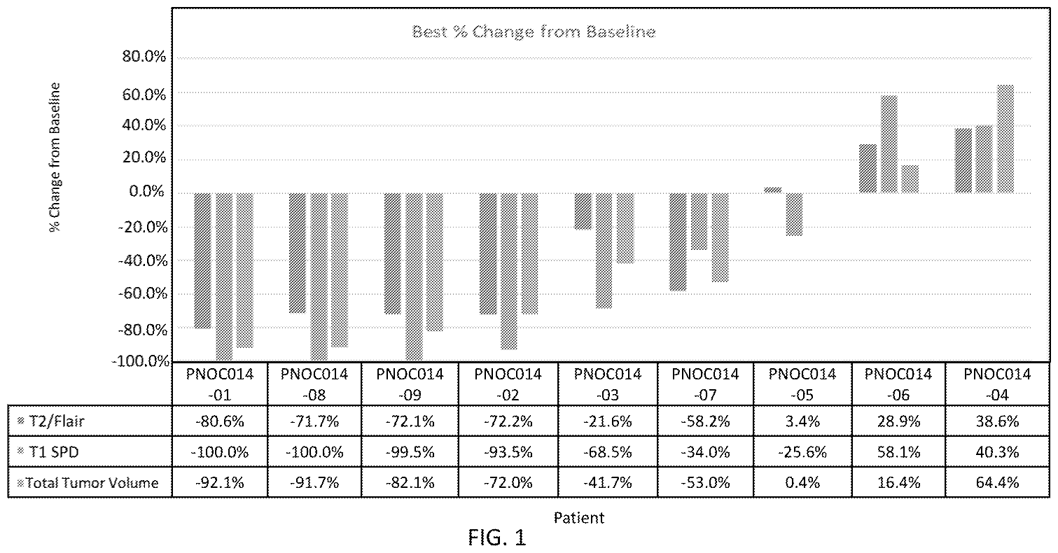

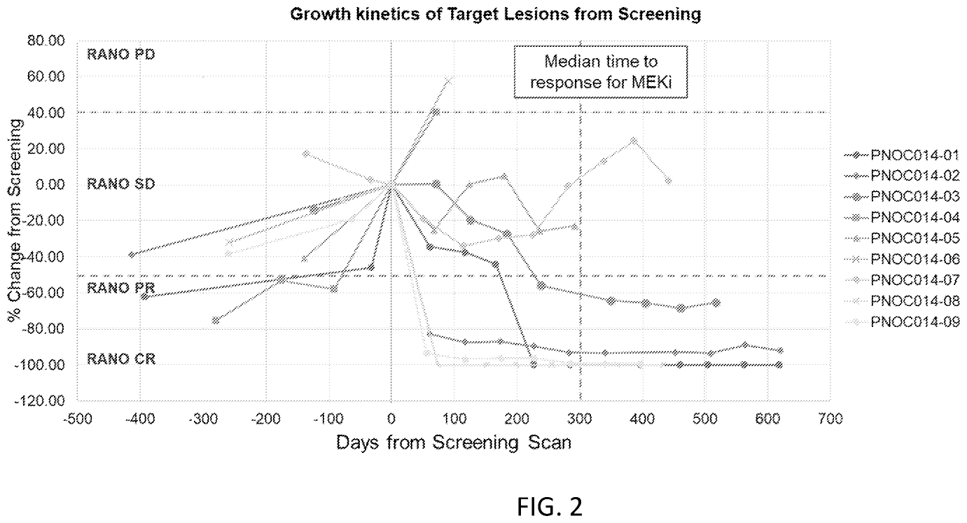

Additional results and analyses are shown in FIG. 1 and FIG. 2.

FIG. 2 illustrates Phase 1 trial data of these nine individual pediatric LGG (pLGG) patient responses to Compound A over time. Shrinkage in lesion size was observed in six of nine patients in the first radiologic images obtained after initiation of Compound A dosing. The median time to response was 10.5 weeks. Two patients achieved a complete response that was maintained throughout the dosing period of up to two years. Three patients had a partial response, two achieved prolonged stable disease, and two did not achieve a response.

FIG. 1 illustrates Phase 1 trial data with these pLGG patients that had a complete (100% reduction) or partial response (>50% reduction in the bi-dimensional measurement of the tumor) to treatment with Compound A. Five of the eight patients with a RAF fusion had either a complete response or a partial response per RANO criteria, defined as ≥50% decrease, compared with baseline. Two of eight patients with a RAF fusion had prolonged stable disease. One patient with a RAF fusion did not respond to Compound A. One patient with an NF1-associated pLGG did not respond to Compound A.

### Example 3: Compound A Treatment Schedule

This trial will follow a modified Bayesian adaptive Sub-TITE design (subgroup-specific time-to-event continual reassessment method) to allow separate determination of the maximum tolerated dose in the two BSA subgroups (see Table 1-C). Compound A will be administered as an oral tablet. The starting dose will be 420 mg/m²/dose by mouth every week (see Table 1-C). Dose escalation decisions will be informed by the Sub-TITE Bayesian model. Pharmacokinetic studies will be performed on all patients of the trial. Pharmacodynamic studies, including measurement of phosphorylated ERK in peripheral blood mononuclear cells, will also be performed on all patients in the trial. Tissue-based pharmacodynamic studies will be performed on patients where tumor tissue is available.

### Example 4: Phase 2 Study to Evaluate the Efficacy and Safety of Compound A in Pediatric and Young Adult Patients with Relapsed or Progression Low-Grade Glioma

This trial will follow a Phase 2, multicenter, open label study to evaluate the safety and efficacy of oral pan-RAF inhibitor Compound A in pediatric, adolescent, and young adult patients with recurrent or progressive low-grade glioma harboring a known BRAF alteration.

Approximately 60 pediatric patient will be treated with Compound A, an oral pan-RAF inhibitor, for a planned period of 26 cycles will be treated with Compound A for a planned period of 26 cycles (approximately 24 months).

Compound A will be administered at the recommended phase 2 dose (RP2D) at 420 mg/m² (not to exceed 600 mg) orally, once per week for each 28-day treatment cycle. Compound A is administered as an oral tablet. For example, immediate-release tablets in 2 strengths, 20 mg and 100 mg, can be administered. Treatment cycles will repeat every 28 days in the absence of disease progression or unacceptable toxicity. Patients will undergo evaluation of their disease at the end of every third cycle. Patients will continue on Compound A until radiographic evidence of disease progression by RANO criteria as determined by treating investigator, unacceptable toxicity, patient withdrawal of consent, or death.

Patients who have radiographic evidence of disease progression may be allowed to continue Compound A if, in the opinion of the investigator and approval of the Sponsor, the patient deriving clinical benefit from continuing study treatment. Disease assessments for patients being treated beyond progression should continue as per regular schedule.

Primary outcome measures will be performed by an independent radiology review committee (IRC) based on RANO criteria. Secondary outcome measures will also be considered, such as:
1) safety and tolerability [Time frame: From first dose to end of treatment],
2) pharmacokinetics [Time frame: from first dose to end of treatment],
3) effect on electrocardiogram (ECG) and QT interval corrected for heart rate by Fridericia's formula (QTcF) prolongation [Timeframe: from first dose to end of treatment],
4) ORR by investigator using RANO criteria [Time Frame: within 12 months of treatment],
) ORR by IRC and Investigator using RAPNO criteria [Time Frame: within 12 months of treatment],
6) Progression free survival (PFS) by IRC and investigator using RANO and RAPNO criteria [Time Frame: From first dose to end of study],
7) Duration of response (DOR) with best overall response of CR or PR using RANO and RAPNO criteria [Time Frame: within 12 months of treatment],
8) Time to response [Time frame: within 12 months of treatment],
9) clinical benefit rate [Time frame: within 12 months of treatment],
10) visual acuity [Time frame: within 12 months of treatment], and
11) molecular profiling [Time Frame: from first dose to end of study].

Other pre-specified outcome measures can be assessed, such as:
1) response and time to progression (TTP) [Time frame: within 12 months of treatment],
2) total tumor volume [time frame: from first dose to end of treatment],
3) change in diffusion coefficients [Time frame: from first dose to end of treatment],
4) quality of life (QoL) and health utilities [Time Frame: from first dose to end of treatment],
5) motor function [time frame: from first dose to end of treatment], and
6) durability of response [time frame: 2 years following end of treatment].

Inclusion criteria can include patients between the ages of 6 months and 25 years of age with relapsed or progressive LGG with known activating BRAF activation. Inclusion criteria can also require confirmation of histopathologic diagnosis of LGG and molecular diagnosis of activating BRAF alternation. In some cases, the patient must have received at least one line of systemic therapy and have evidence of radiographic progression. In some cases, the patient must have at least 1 measurable lesion as defined by RANO criteria.

In some cases, patients may be excluded if the tumor has additional previously-known activating molecular alterations. Patients may be excluded if there are symptoms of clinical progression in the absence of radiographic progression. In some cases, patients may be excluded if there are known or suspected diagnosis of neurofibromatosis type 1 (NF-1). Further, patients may be excluded according to trial protocols.

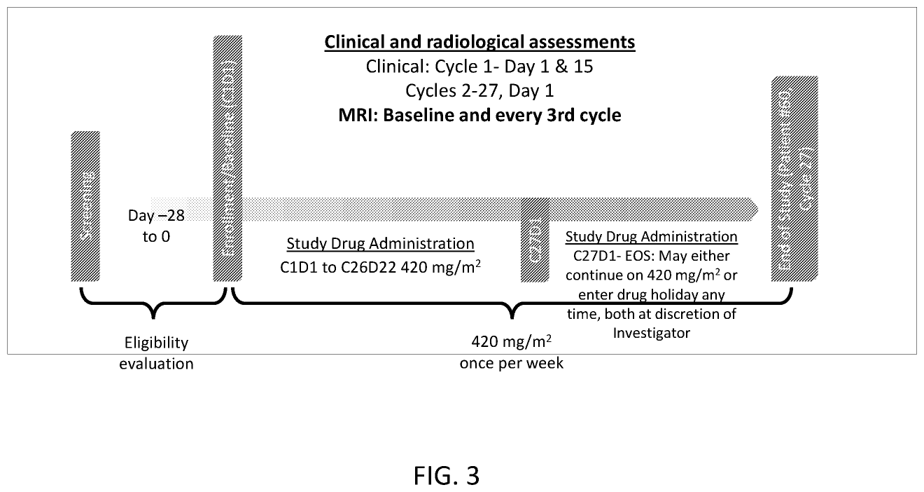

An exemplary trial design is illustrated in FIG. 3.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a low grade glioma (LGG) in a subject in need thereof comprising,
administering (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof to the subject, wherein an initial dose of the Compound A, or a pharmaceutically acceptable salt thereof is equivalent to about 400 mg/m² to about 600 mg/m² of Compound A per week,
wherein the subject is less than 20 years of age.

2. The method of claim 1, wherein the initial dose of the Compound A, or a pharmaceutically acceptable salt thereof is equivalent to about 500 mg/m² to about 600 mg/m² of Compound A per week.

3. The method of claim 1, wherein the initial dose of the Compound A, or a pharmaceutically acceptable salt thereof is equivalent to about 400 mg/m² to about 500 mg/m² of Compound A per week.

4. The method of claim 1, wherein the initial dose of the Compound A, or a pharmaceutically acceptable salt thereof is equivalent to about 420 mg/m² of Compound A per week.

5. The method of claim 1, wherein the initial dose of the Compound A, or a pharmaceutically acceptable salt thereof is equivalent to about 530 mg/m² of Compound A per week.

6. A method of treating a low grade glioma (LGG) in a subject in need thereof comprising,
administering to the subject (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof in an amount sufficient to achieve in the subject a maximum observed blood plasma concentration (Cmax) of Compound A of at least 2000 ng/mL,
wherein the subject is less than 20 years of age.

7. The method of claim 6, wherein the Compound A or a pharmaceutically acceptable salt thereof is administered in an amount sufficient to achieve in the subject a Cmax of Compound A of 2000 ng/mL to 8000 ng/mL.

8. A method of treating a low grade glioma (LGG) in a subject in need thereof comprising,
administering to the subject (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof in an amount sufficient to achieve an area under the concentration curve (AUCₛₛ) of Compound A of at least about 400,000 ng*h/mL,
wherein the subject is less than 20 years of age.

9. The method of claim 8, wherein the Compound A or a pharmaceutically acceptable salt thereof is administered in an amount sufficient to achieve in the subject an (AUCₛₛ) of Compound A of 400,000 ng*h/ml to 1600,000 ng*h/ml.

10. A method of treating a low grade glioma (LGG) in a subject in need thereof comprising:
administering to the subject (i) (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof, in combination with (ii) one or more therapeutic agents for treating a skin-related condition or disorder.
wherein the subject is less than 20 years of age.

11. The method of claim 10, wherein the one or more therapeutic agents are administered on pigmented skin.

12. The method of any one of claims 1 to 11, wherein the LGG is a radiographically recurrent or radiographically progressive disease.

13. The method of any one of claims 1 to 12, wherein the Compound A or a pharmaceutically acceptable salt thereof is (R)-2-(1-(6-amino-5-chloropyrimidine-4-carboxamido)ethyl)-N-(5-chloro-4-(trifluoromethyl)pyridin-2-yl)thiazole-5-carboxamide.

14. The method of any one of claims 1 to 13, wherein the Compound A or a pharmaceutically acceptable salt thereof is administered as a liquid suspension.

15. The method of any one of claims 1 to 13, wherein the Compound A or a pharmaceutically acceptable salt thereof is administered as a tablet.

16. The method of any one of claims 1 to 15, wherein the Compound A or a pharmaceutically acceptable salt thereof is administered as a single dose per week.

17. The method of any one of claims 1 to 15, wherein the Compound A or a pharmaceutically acceptable salt thereof is administered 2-4 doses a week.

18. The method of any one of claims 1 to 17, wherein the Compound A or a pharmaceutically acceptable salt thereof is administered for a period of at least 24 months.

19. The method of any one of claims 1 to 18, wherein the subject is 20 years of age or less.

20. The method of any one of claims 1 to 18, wherein the subject is 15 years of age or less.

21. The method of any one of claims 1 to 20, wherein the subject has a body surface area (BSA) of from 0.5 m² to about 2.0 m².

22. The method of any one of claims 1 to 20, wherein the subject has a BSA of from 0.5 m² to about 1.5 m².

23. The method of any one of claims 1 to 22, wherein the LGG has one or more of the following mutations: RAS positive mutation, RAF positive mutation, MEK positive mutation, and ERK positive mutation.

24. The method of any one of claims 1 to 22, wherein the LGG has a BRAF mutation.

25. The method of claim 24, wherein the BRAF mutation is a non-V600 BRAF mutation.

26. The method of claim 24, wherein the BRAF mutation is V600E mutation.

27. The method of any one of claims 1 to 26, wherein the subject is identified having one or more of the following wild-type fusions: KIAA1549:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF21:BRAF, FXR1:BRAF, RNF130:BRAF, BRAF:MACF1, TMEM106B:BRAF, PPC1CC:BRAF, CUX1:BRAF, SRGAP3:RAF1, QK1:RAF1, FYCO:RAF1, ATG7:RAF1, and NFIA:RAF1.

28. The method of any one of claims 1 to 26, wherein the subject is identified having KIAA1549:BRAF wild-type fusion.

29. The method of any one of claims 1 to 28, wherein the subject has received one or more therapies selected from surgery, radiation, and chemotherapy before the administering of Compound A, or a pharmaceutically acceptable salt thereof.

30. The method of claim 29, wherein the subject has received a complete or partial resection before the administering of Compound A, or a pharmaceutically acceptable salt thereof.

31. The method of any one of the preceding claims, wherein the Compound A or a pharmaceutically acceptable salt thereof is administered at a maximum dose of 600 mg.

32. The method of claim 31, wherein the maximum dose is 600 mg orally (PO) once a week.
